SEQUENCE LISTING (48 sequences)

SEQ ID NO: 1 - protein, 9 aa, Alphainfluenzavirus influenzae
ILRGSVAHK

SEQ ID NO: 2 - protein, 9 aa, Alphainfluenzavirus influenzae
ELRSRYWAI

SEQ ID NO: 3 - protein, 9 aa, Alphainfluenzavirus influenzae
SRYWAIRTR

SEQ ID NO: 4 - protein, 9 aa, Alphainfluenzavirus influenzae
CTELKLSDY

SEQ ID NO: 5 - protein, 9 aa, Alphainfluenzavirus influenzae
GILGFVFTL

SEQ ID NO: 6 - protein, 9 aa, Alphainfluenzavirus influenzae
SIIPSGPLK

SEQ ID NO: 7 - protein, 8 aa, Alphainfluenzavirus influenzae
ASCMGLIY

SEQ ID NO: 8 - protein, 9 aa, Alphainfluenzavirus influenzae
FMYSDFHFI

SEQ ID NO: 9 - protein, 9 aa, Alphainfluenzavirus influenzae
FVRQCFNPM

SEQ ID NO: 10 - protein, 9 aa, Alphainfluenzavirus influenzae
VSDGGPNLY

SEQ ID NO: 11 - protein, 9 aa, Alphainfluenzavirus influenzae
FLKDVMESM

SEQ ID NO: 12 - protein, 9 aa, Alphainfluenzavirus influenzae
NMLSTVLGV

SEQ ID NO: 13 - protein, 9 aa, Alphainfluenzavirus influenzae
MMMGMFNML

SEQ ID NO: 14 - protein, 9 aa, Alphainfluenzavirus influenzae
YSHGTGTGY

SEQ ID NO: 15 - protein, 9 aa, Alphainfluenzavirus influenzae
HSNLNDATY

SEQ ID NO: 16 - protein, 11 aa, Alphainfluenzavirus influenzae
RRSGAAGAAVK

SEQ ID NO: 17 - protein, 9 aa, Alphainfluenzavirus influenzae
LLTEVETYV

SEQ ID NO: 18 - protein, 9 aa, Alphainfluenzavirus influenzae
MVLASTTAK

SEQ ID NO: 19 - protein, 9 aa, Alphainfluenzavirus influenzae
RGINDRNFW

SEQ ID NO: 20 - protein, 9 aa, Alphainfluenzavirus influenzae
FLLMDALKL

SEQ ID NO: 21 - protein, 9 aa, Alphainfluenzavirus influenzae
YPYDVPDYA

SEQ ID NO: 22 - DNA, 27 bp, synthetic construct
  source 1..27: mol_type = other DNA
atattgagagggtcggttgctcacaag

SEQ ID NO: 23 - DNA, 27 bp, synthetic construct
  source 1..27: mol_type = other DNA
gaactgagaagcaggtactgggccata

SEQ ID NO: 24 - DNA, 27 bp, synthetic construct
  source 1..27: mol_type = other DNA
agcaggtactgggccataaggaccaga

SEQ ID NO: 25 - DNA, 27 bp, synthetic construct
  source 1..27: mol_type = other DNA
tgcaccgaactcaaactcagtgattat

SEQ ID NO: 26 - DNA, 27 bp, synthetic construct
  source 1..27: mol_type = other DNA
gggattttaggatttgtgttcacgctc

SEQ ID NO: 27 - DNA, 27 bp, synthetic construct
  source 1..27: mol_type = other DNA
tctatcatcccgtcaggccccctcaaa

SEQ ID NO: 28 - DNA, 24 bp, synthetic construct
  source 1..24: mol_type = other DNA
gccagttgtatgggcctcatatac

SEQ ID NO: 29 - DNA, 27 bp, synthetic construct
  source 1..27: mol_type = other DNA
ttcatgtattcagattttcacttcatc

SEQ ID NO: 30 - DNA, 27 bp, synthetic construct
  source 1..27: mol_type = other DNA
tttgtgcgacaatgcttcaatccgatg

SEQ ID NO: 31 - DNA, 27 bp, synthetic construct
  source 1..27: mol_type = other DNA
gtctccgacggaggcccaaatttatac

SEQ ID NO: 32 - DNA, 27 bp, synthetic construct
  source 1..27: mol_type = other DNA
ttccttaaggatgtaatggagtcaatg

SEQ ID NO: 33 - DNA, 27 bp, synthetic construct
  source 1..27: mol_type = other DNA
aatatgttaagcactgtattaggcgtc

SEQ ID NO: 34 - DNA, 27 bp, synthetic construct
  source 1..27: mol_type = other DNA
atgatgatgggcatgttcaatatgtta

SEQ ID NO: 35 - DNA, 27 bp, synthetic construct
  source 1..27: mol_type = other DNA
tacagccatgggacaggaacaggatac

SEQ ID NO: 36 - DNA, 27 bp, synthetic construct
  source 1..27: mol_type = other DNA
cattccaatttgaatgatgcaacttat

SEQ ID NO: 37 - DNA, 33 bp, synthetic construct
  source 1..33: mol_type = other DNA
aggaggtctggagccgcaggtgctgcagtcaaa

SEQ ID NO: 38 - DNA, 27 bp, synthetic construct
  source 1..27: mol_type = other DNA
cttctaaccgaggtcgaaacgtacgta

SEQ ID NO: 39 - DNA, 27 bp, synthetic construct
  source 1..27: mol_type = other DNA
atggttttagccagcactacagctaag

SEQ ID NO: 40 - DNA, 27 bp, synthetic construct
  source 1..27: mol_type = other DNA
cgtgggatcaatgatcggaacttctgg

SEQ ID NO: 41 - DNA, 27 bp, synthetic construct
  source 1..27: mol_type = other DNA
ttcctgctgatggatgccttaaaatta

SEQ ID NO: 42 - DNA, 27 bp, synthetic construct
  source 1..27: mol_type = other DNA
tacccatacgatgttccagattacgct

SEQ ID NO: 43 - DNA, 225 bp, unidentified
  source 1..225: mol_type = other DNA
cagatcttcgtgaagactctgactggtaagaccatcaccctcgaggttgagcccagtgacaccatcgagaatgtcaaggcaaagatccaagataaggaaggcatccctcctgaccagcagaggctgatctttgctggaaaacagctggaagatgggcgcaccctgtctgactacaacatccagaaagagtccaccctgcacctggtgctccgtctcagaggtgta

SEQ ID NO: 44 - DNA, 821 bp, synthetic construct
  source 1..821: mol_type = other DNA
ggtaccggatccgccgccaccatgagtcttctaaccgaggtcgaaacgtacgtactctctatcatcccgtcaggccctctcaaagccgagatcgcacagagacttgaagatgtctttgcagggaagaacaccgatcttgaggttctcatggaatggctaaagacaagaccaatcctgtcacctctgactaagggtattttaggatttgtgttcacgctcaccgtgcccagtgagcgaggactgcagcgtagacgctttgtccaaaatgcccttaatgggaacggtgatccaaataacatggacaaagcagttaaactgtataggaagctcaagagggagataacattccatggtgccaaagaaatctcactcagttattctgctggtgcacttgccagttgtatgggcctcatatacaacaggatgggtgctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcctatcagaaacgaatgggtgtgcagatgcaacggttcaagtacccatacgatgttccagattacgcttaagtttaaacctcgag

SEQ ID NO: 45 - DNA, 782 bp, synthetic construct
  source 1..782: mol_type = other DNA
ggtaccggatccgccgccaccatgatattgagagggtcggttgctcacaaggcggcgtatttcctgctgatggatgccttaaaattagcggcgtatagcaggtactgggccataaggaccagagcggcgtattgcaccgaactcaaactcagtgattatgcggcgtatgggattttaggatttgtgttcacgctcgcggcgtattctatcatcccgtcaggccctctcaaagcggcgtatgccagttgtatgggcctcatatacgcggcgtatttcatgtattcagattttcacttcatcgcggcgtattttgtgcgacaatgcttcaatccgatggcggcgtatgtctccgacggaggcccaaatttatacgcggcgtatttccttaaggatgtaatggagtcaatggcggcgtatcgtgggatcaatgatcggaacttctgggcggcgtatatgatgatgggcatgttcaatatgttagcggcgtattacagccatgggacaggaacaggatacgcggcgtatcattccaatttgaatgatgcaacttatgcggcgtataggaggtctggagccgcaggtgctgcagtcaaagcggcgtatcttctaaccgaggtcgaaacgtacgtagcggcgtatatggttttagccagcactacagctaaggcggcgtataatatgttaagcactgtattaggcgtcgcggcgtatgaactgagaagcaggtactgggccatatacccatacgatgttccagattacgcttaagtttaaacctcgag

SEQ ID NO: 46 - DNA, 611 bp, synthetic construct
  source 1..611: mol_type = other DNA
ggtaccggatccgccgccaccatgatattgagagggtcggttgctcacaagttcctgctgatggatgccttaaaattaagcaggtactgggccataaggaccagatgcaccgaactcaaactcagtgattatgggattttaggatttgtgttcacgctctctatcatcccgtcaggccctctcaaagccagttgtatgggcctcatatacttcatgtattcagattttcacttcatctttgtgcgacaatgcttcaatccgatggtctccgacggaggcccaaatttatacttccttaaggatgtaatggagtcaatgcgtgggatcaatgatcggaacttctggatgatgatgggcatgttcaatatgttatacagccatgggacaggaacaggataccattccaatttgaatgatgcaacttataggaggtctggagccgcaggtgctgcagtcaaacttctaaccgaggtcgaaacgtacgtaatggttttagccagcactacagctaagaatatgttaagcactgtattaggcgtcgaactgagaagcaggtactgggccatatacccatacgatgttccagattacgcttaagtttaaacctcgag

SEQ ID NO: 47 - DNA, 835 bp, synthetic construct
  source 1..835: mol_type = other DNA
ggtaccggatccgccgccaccatgcagatcttcgtgaagactctgactggtaagaccatcaccctagaggttgagcccagtgacaccatcgagaatgtcaaggcaaagatccaagataaggaaggcatccctcctgaccagcagaggctgatctttgctggaaaacagctggaagatgggcgcaccctgtctgactacaacatccagaaagagtccaccctgcacctggtgctccgtctcagaggtgtatattgagagggtcggttgctcacaagttcctgctgatggatgccttaaaattaagcaggtactgggccataaggaccagatgcaccgaactcaaactcagtgattatgggattttaggatttgtgttcacgctctctatcatcccgtcaggccctctcaaagccagttgtatgggcctcatatacttcatgtattcagattttcacttcatctttgtgcgacaatgcttcaatccgatggtctccgacggaggcccaaatttatacttccttaaggatgtaatggagtcaatgcgtgggatcaatgatcggaacttctggatgatgatgggcatgttcaatatgttatacagccatgggacaggaacaggataccattccaatttgaatgatgcaacttataggaggtctggagccgcaggtgctgcagtcaaacttctaaccgaggtcgaaacgtacgtaatggttttagccagcactacagctaagaatatgttaagcactgtattaggcgtcgaactgagaagcaggtactgggccatatacccatacgatgttccagattacgcttaagtttaaacctcgag

SEQ ID NO: 48 - DNA, 1007 bp, synthetic construct
  source 1..1007: mol_type = other DNA
ggtaccggatccgccgccaccatgcagatcttcgtgaagactctgactggtaagaccatcaccctagaggttgagcccagtgacaccatcgagaatgtcaaggcaaagatccaagataaggaaggcatccctcctgaccagcagaggctgatctttgctggaaaacagctggaagatgggcgcaccctgtctgactacaacatccagaaagagtccaccctgcacctggtgctccgtctcagaggtgtaatattgagagggtcggttgctcacaaggcggcgtatttcctgctgatggatgccttaaaattagcggcgtatagcaggtactgggccataaggaccagagcggcgtattgcaccgaactcaaactcagtgattatgcggcgtatgggattttaggatttgtgttcacgctcgcggcgtattctatcatcccgtcaggccctctcaaagcggcgtatgccagttgtatgggcctcatatacgcggcgtatttcatgtattcagattttcacttcatcgcggcgtattttgtgcgacaatgcttcaatccgatggcggcgtatgtctccgacggaggcccaaatttatacgcggcgtatttccttaaggatgtaatggagtcaatggcggcgtatcgtgggatcaatgatcggaacttctgggcggcgtatatgatgatgggcatgttcaatatgttagcggcgtattacagccatgggacaggaacaggatacgcggcgtatcattccaatttgaatgatgcaacttatgcggcgtataggaggtctggagccgcaggtgctgcagtcaaagcggcgtatcttctaaccgaggtcgaaacgtacgtagcggcgtatatggttttagccagcactacagctaaggcggcgtataatatgttaagcactgtattaggcgtcgcggcgtatgaactgagaagcaggtactgggccatatacccatacgatgttccagattacgcttaagtttaaacctcgag

(19) United States
(12) Patent Application Publication
(10) Pub. No.: US 2026/0124292 A1
(43) Pub. Date: May 7, 2026

(54) MULTIEPITOPE UNIVERSAL INFLUENZA VACCINE

(71) Applicant: UNIVERSITY OF VETERINARY MEDICINE HANNOVER, FOUNDATION, Hannover (DE)

(72) Inventors: Sharmistha Dam, Hannover (DE); Gustaaf Frank Rimmelzwaan, Hannover (DE); Albertus Dominicus Marcellinus Erasmus Osterhaus, Amsterdam (NL)

(21) Appl. No.: 19/114,661

(22) PCT Filed: Sep. 22, 2023
(86) PCT No.: PCT/EP2023/076265

§ 371 Date: Mar. 24, 2025

(30) Foreign Application Priority Data
Sep. 23, 2022 (EP) 22197406.6

(51) Int. Cl. A61K 39/145; A61K 39/00; A61P 37/04; C07K 14/005

(52) U.S. Cl. CPC A61K 39/145; A61P 37/04; C07K 14/005; A61K 2039/53; A61K 2039/70; C07K 2319/95; C12N 2760/16122; C12N 2760/16134; C12N 2760/16171

(57) ABSTRACT

Polyepitope vaccine formulations relating to at least 5 of influenza A virus (IAV) derived peptides capable of inducing IFNγ by CD8+ T cells selected from the group of peptides with amino acid sequences SEQ ID NO:1 (ILRGSVAHK), SEQ ID NO:2 (ELRSRYWAI), SEQ ID NO:3 (SRYWAIRTR), SEQ ID NO:4 (CTELKLSDY), SEQ ID NO:5 (GILGFVFTL), SEQ ID NO:6 (SIIPSGPLK), SEQ ID NO:7 (ASCMGLIY), SEQ ID NO:8 (FMYSDFHFI), SEQ ID NO:9 (FVRQCFNPM), SEQ ID NO:10 (VSDGGPNLY), SEQ ID NO:11 (FLKDVMESM), SEQ ID NO:12 (NMLSTVLGV), SEQ ID NO:13 (MMMGMFNML), SEQ ID NO:14 (YSHGTGTGY), SEQ ID NO:15 (HSNLNDATY), SEQ ID NO:16 (RRSGAAGAAVK), SEQ ID NO:17 (LLTEVETYV), SEQ ID NO:18 (MVLASTTAK), SEQ ID NO:19 (RGINDRNFW) and SEQ ID NO:20 (FLLMDALKL).

### CROSS-REFERENCE TO RELATED APPLICATIONS

This application is a national phase entry under 35 U.S.C. § 371 of International Patent Application PCT/EP2023/076265, filed Sep. 22, 2023, designating the United States of America and published as International Patent Publication WO 2024/062106 A1 on Mar. 28, 2024, which claims the benefit under Article 8 of the Patent Cooperation Treaty of European Patent Application Serial No. 22197406.6, filed Sep. 23, 2022.

### TECHNICAL FIELD

The disclosure relates to the field of vaccination against influenza virus infections with broad spectrum influenza vaccines.

### BACKGROUND

Influenza is a common acute respiratory disease due to a virus that causes annual seasonal epidemics. Three major pandemics occurred in the 20th century, 1918-1919, 1957 and 1968, and one in 2009, mainly due to genetic variants of type A influenza virus (IAV). In temperate regions the incidence of hospitalization increases during annual influenza epidemics. More than 90% of deaths linked to influenza involve people over 65 years of age. Yearly vaccination is the main preventive measure in this age group. Yearly vaccination is also recommended for younger people with serious chronic disease. Preferably, the vaccine strains closely match the epidemic strains circulating at the time of vaccination antigenically to afford optimal protection. In relative terms, vaccination of people over 65 reduces the number of deaths linked to influenza by up to 80%, hospitalization and pneumonia by up to 50%, and symptomatic influenza by up to 30%. The full impact of influenza is increasingly recognized as an illness that goes well beyond pneumonia and influenza statistics. Peak months of mortality due to respiratory illness, ischemic heart disease, cerebrovascular events, and diabetes in adults 70 years and older coincide with annual influenza epidemics, suggesting that influenza illness is the major cause of excess mortality in this population during the winter months (McElhaney 2011). Vaccination programs using the current split-virus vaccines are cost-saving in the over 65 population even though these vaccines often fail to provide adequate protection in older adults and influenza illness continues to have devastating consequences in this population. Rising rates of hospitalization are anticipated from seasonal influenza and, in the event of a real pandemic in older people, threaten to paralyze the health and social systems of support. Development of vaccines is a priority to prepare for potential pandemics but is complicated by antigenic variation of the surface glycoprotein hemagglutinin (de Vries et al. 2018).

Vaccination is the most appropriate countermeasure to control the spread of IAV and prevent disease. However, the lack of an efficacious method to predict the circulating strains sometimes leads to a mismatch between the annual vaccine and circulating viruses. To illustrate, the sudden emergence of the 2009 influenza A/H1N1 pandemic strain and the recent appearance of the highly pathogenic avian A/H7N9 strain in 2013 in China, underscores the unpredictable nature of the virus and the difficulty in comprehending the emergence of new strains. Two types of influenza vaccine are widely available: inactivated influenza vaccines (IIV) and live attenuated influenza vaccines (LAIV). Traditionally, to cope with yearly varying antigenic variation, influenza vaccines (both IIV and LAIV) have been produced to protect against 3 or 4 different seasonal influenza viruses (also called trivalent or quadrivalent vaccines). Current quadrivalent vaccines contain components of influenza A(H3N2), A(H1N1) and 2 influenza lineages of influenza B virus. The trivalent or quadrivalent vaccines foremost rely on specific targeting of the major surface protein, hemagglutinin (HA), and are standardized as stimulating anti-HA antibodies as the primary correlate of protection, but these vaccines have limited and somewhat unpredictable efficacy, particularly in those that most require protection such as the elderly, young, and infirm. Regardless of the type or composition of seasonal influenza vaccine, vaccination needs be administered annually to provide optimal protection against infection.

As influenza vaccine effectiveness can vary yearly due to the constant evolving nature of influenza viruses, including those circulating and infecting humans, these vaccines are updated regularly to antigenically match which influenza strains that are in circulation. The WHO organizes regular consultations with an advisory group of experts gathered from WHO Collaboration Centers and WHO Essential Regulatory Laboratories to analyze influenza virus surveillance data generated by the WHO Global Influenza Surveillance and Response System. The recommendations issued are used by the national vaccine regulatory agencies and pharmaceutical companies to develop, produce, and license influenza vaccines for the following influenza season. Because of the evolving nature, the protection provided by a vaccine may still vary from season to season and depends in part on the age and health status of the person getting the vaccine and the factual similarity or match between the viruses in the vaccine and those in circulation. During years when the vaccine match is good, it is possible to measure substantial benefits from vaccination in terms of preventing illness and complications after infection with the virus. However, the benefits of yearly influenza vaccination will still vary, depending on characteristics of the person being vaccinated (for example, their health and age), what influenza viruses are circulating that season and, potentially, which type of vaccine is used. Indeed, some people may experience symptoms despite getting vaccinated, for example, because they may have been exposed to a virus that is different from the viruses used in the vaccine. The ability of an influenza vaccine to protect a person depends largely on the match or mismatch between the vaccine viruses chosen to make the vaccine used in that person and those viruses spreading and causing illness in that person. There are many different influenza viruses that spread and cause illness among people. During years when the vaccine match is generally bad, substantially reduced benefits from vaccination in terms of preventing influenza illness and complications are to be expected, again risking the elderly most (McElhaney 2011).

To alleviate the mismatch problems, some have suggested to use anti-viral drugs such as amantadine, zanamivir and oseltamivir in influenza as an adjunct to vaccination in some situations (‘Antiviral drugs in influenza: an adjunct to vaccination in some situations’ 2006). In practice, however, antiviral drugs are not an alternative to influenza vaccination but may be a useful adjunct only in some situations. It is best to limit their use to short-term prophylaxis of vulnerable persons in situations where the risk of contracting influenza virus infection is high. However, betting on anti-viral therapy employing anti-viral drugs negates the fact that the infections are due to the mismatch, it may thus be better to improve on the mismatch instead by providing better matching vaccines in the first place.

Furthermore, it is now well recognized that older people respond less well to immunization protocols. Protection against influenza by vaccination with hemagglutinins is the prototype example. Despite programs that have raised vaccination rates dramatically over the past three decades, influenza remains a major cause of morbidity and mortality in the elderly. This, in part, is because vaccine responses are reduced in older recipients. Some have suggested to improve the yearly vaccine response by using thymosin alpha 1 as an adjunct to influenza vaccination to activate T-cell responses in the elderly (Ershler, Gravenstein, and Geloo 2007), however, this has not gained practical ground.

Another approach to improve influenza vaccines is to include adjuvants; substances that boost the immune response. Adjuvants are particularly beneficial for influenza vaccines administered during a pandemic when a rapid response is required or for use in patients with impaired immune responses, such as infants and the elderly. Tregoning et al., (Tregoning, Russell, and Kinnear 2018), outline the current use of adjuvants in human influenza vaccines, including what they are, why they are used and what is known of their mechanism of action. To date, six adjuvants have been used in licensed human vaccines: Alum, MF59, AS03, AF03, virosomes and heat labile enterotoxin (LT).

Yet others aim to deviate from yearly selecting different vaccine viruses and aim at immunization using universal influenza vaccines for protection against all or most influenza sub-types. The fundamental principle of such desired universal influenza vaccines is based on conserved antigens found in most influenza strains, such as matrix 2, nucleocapsid, matrix 1 and stem of hemagglutinin proteins. These antigens may trigger cross-protective immunity against different influenza strains. Many researchers have attempted to produce the conserved epitopes of these antigens in the form of peptides in the hope of generating universal influenza vaccine candidates that can broadly induce cross-reactive protection against influenza viral infections, however, up until now such efforts have largely failed. Hence, various strategies, such as combining peptides as multi-epitope vaccines or presenting peptides through vaccinia virus particles, are subjected to clinical trials (Romeli, Hassan, and Yap 2020).

Sheik et al. (Bioinformatics. 2016 Nov. 1; 32(21):3233-3239) is an in-silico exercise purportedly to help design vaccines, albeit lacking in vitro or in vivo data that may show antigenicity or immunogenicity of any of its suggestions. Despite often being used interchangeably, the terms immunogenicity and antigenicity have distinct meaning. The term immunogenicity refers to the ability of a substance to help induce cells of the immune system elicit a cellular and/or humoral immune response, while antigenicity is the ability to be specifically recognized by an immune response or immunological activities toward the given substance. While all immunogenic substances are by default antigenic, not all antigenic substances are immunogenic, neither need they be immunogenic in different hosts, such as mice or man, or in men or women of various genetic background. In particular, MHC I processing of proteins or polypeptides is depended on the genetic background (i.e., HLA haplotype) of the host, rendering some antigenic substances immunogenic in one, and non-immunogenic in another host. To complicate matters, often the immune response is depended on specific proteolytic processing of antigenic polypeptides in the proteasome of lysosome, to arrive at the desired immunogenic substance. To help facilitate such processing or degradation, often cells attach a degradation signal (degron) to such polypeptides to accommodate routing to the proteasome or lysosome. In line with the above, antigenic determinant is herein defined as a site on the surface of an antigen molecule to which a single antibody molecule or T-cell binds, generally, an antigen has several or many different antigenic determinants and reacts with many different antibodies or cells. An antigenic determinant is also called epitope. An immunogenic determinant is the part of an immunogenic molecule that interacts with a T-cell in triggering or eliciting an immune response, and thus reflects on the active potential of a host to induce the immune response per se. Sheik et al. designed but not constructed nor tested the two epitope ensemble vaccine designs comprising (in itself antigenic) influenza A epitopes as putative non-seasonal influenza vaccines; one specifically targets the US population and the other is meant to be a universal vaccine. The purportedly USA-specific vaccine comprised 6 CD8+ T cell epitopes (among which SEQ ID NO:5 (GILGFVFTL) and SEQ ID NO:8 (FMYSDFHFI) and 3 CD4+ epitopes. The purportedly universal vaccine comprised 8 CD8+ epitopes: (among which SEQ ID NO:1 (ILRGSVAHK), SEQ ID NO:5 (GILGFVFT), SEQ ID NO:8 (FMYSDFHFI), SEQ ID NO:10 (VSDGGPNLY) and SEQ ID NO:14 (YSHGTGTGY) and again the 3 CD4+ epitopes. It is suggested that epitopes can be delivered as poly-epitope peptide(s) or as part of a viral vector. No spacer or linker sequences were attached or provided, and no degron such as ubiquitin was attached or provided with the construct designs. Whether the ensembled designs of Sheik et al. bear sufficient antigenicity to be recognized by cells of the immune system or even can or cannot be used as immunogenic substances to mount the desired immune response required for an actual vaccine substance is not disclosed in Sheik et al.

Nielsen et al. (Journal of Immunological Methods 360 (2010) 149-156), are not aiming at developing a vaccine but at developing methods to confirm the presence and capacity to detect CEF virus specific CD8+ T cells in PBMC. For that purpose, Nielsen et al. provide an antigenic combination of 32 different HLA-1 restricted epitopes of the widely used CEF (Cytomegalovirus, Epstein-Barr Virus and Influenza Virus) positive control peptide pool in a single polyepitope construct, (hence its name CEF-polyepitope 32 construct) as presented by mRNA, (among which epitopes are found SEQ ID NO:1 (ILRGSVAHK), SEQ ID NO:2 (ELRSRYWAI), SEQ ID NO:3 (SRYWAIRTR), SEQ ID NO:4 (CTELKLSDY), SEQ ID NO:5 (GILGFVFTL), SEQ ID NO:6 (SIIPSGPLK), SEQ ID NO:7 (ASCMGLIY), SEQ ID NO:8 (FMYSDFHFI), and SEQ ID NO:10 (VSDGGPNLY)) and to be used as a positive control pool antigen to test the general presence of CEF virus-specific CD8 T cells. No particular spacer or linker sequences were provided, CEF-construct epitopes were presented in their plain viral sequence context, with 3-amino acid heterologous endogenous flanking sequences of the respective epitopes N- as well as C-terminally, thereby increasing the risk of neo-epitope formation instead of reducing it with appropriate spacers or linkers. No ubiquitin was attached or provided with the construct. The extent of interferon-gamma (IFNγ) secretion by influenza virus specific CD8+-CTL of the influenza virus derived epitopes is not provided, and neither was the extent of conservedness of antigenicity of all the epitopes in the pool investigated by Nielsen et al. The purpose of Nielsen et al. was to provide an antigenic determinant tool for assessing MHC class I processing, regardless of HLA haplotype but not to develop a universal influenza vaccine. Indeed, whether the diagnostic tool in itself bears immunogenicity or not is not disclosed in Nielsen et al.

For another example of the many epitope suggestions that have obliterated understanding of the field without providing sufficient guidance to arrive at the desired universal vaccine, recently (Sharma et al. 2021) suggested an in-silico conceptual framework to arrive at a multi-epitope influenza subunit vaccine based on the highly conserved antigenic determinant or epitopic sequences of rapidly evolving (HA), a moderately evolving (NP) and slow evolving (M1) proteins of the virus. The vaccine grand design includes 2 peptide adjuvants, 26 cytotoxic T-cell (CTL) epitopes of a diverse nature, 9 helper T-cell (HTL) epitopes, and 7 linear B-cell lymphocytes (BCL) epitopes to induce innate, cellular, and humoral immune responses against influenza A viruses, of which 3 of the 26 CTL epitopes with SEQ ID NO:5 (GILGFVFTL), SEQ ID NO:17 (LLTEVETYV) and SEQ ID NO:18 (MVLASTTAK) are found in M1 and 1 of the 26, SEQ ID NO:15 (HSNLNDATY), is found in NP. No degron such as ubiquitin is attached or provided. Again, this concept needs experimental validation. The required experimental work may include the synthesis of the, cumbersomely large, designed subunit vaccine followed by the in vitro and in vivo analyses to determine the immunogenicity and delivery of the same for inducing a protective immune response. Formulation, product stability, safety, and protocols for immunizing with such a large vaccine construct are also important aspects to be considered. In short, whether the ensembled designs of Sharma et al. bear sufficient antigenicity to be recognized by cells of the immune system or (better) even can or cannot be used as immunogenic substances to mount the desired immune response required for an actual vaccine substance is not disclosed in Sharma et al. (see also Table 9 herein).

Multimeric-001 (M-001) is an example of a synthetic peptide vaccine that is actually produced and based on nine conserved immunogenic epitopes from HA, NP and M1 proteins of influenza type A and B strains. The M-001 vaccine consists of 3 repetitions of 9 conserved linear epitopes that are prepared as a single recombinant protein. These epitopes are thought to induce humoral as well as cellular immune responses (Atsmon et al. 2012). This peptide-based universal influenza vaccine candidate purportedly inducing T cell-immunity has completed phase I and II trials but has failed to demonstrate any efficacy via a primary outcome study (Atsmon et al. 2012; van Doorn et al. 2017). Indeed, the ability of peptide vaccines to alter and diminish the functionality of T cells, in particular, T regulatory cells (Tregs), has been previously observed (Leggatt 2014), and data from various studies demonstrate that low peptide doses induce high T cell avidity while high or repeat peptide concentrations may favor low avidity T cells and inhibition of the T cell proliferative response (Corradin, Etlinger, and Chiller 1977) required to induce vaccine efficacy. This may also affect the available T cell repertoire in vivo and subsequent pathogen clearance. Such high and low zone tolerance after immunization with different doses of antigen exists is typically thought to require empirical determination (Corradin, Etlinger, and Chiller 1977).

Stambas et al. (Pharmacol Ther. 2008 November; 120(2):186-96) et al. review the understanding of influenza virus-specific CD8+ T cell immunity in experimental mouse models and humans. The characteristics and nature of CD8+ T cell killing are discussed, as is the selection and maintenance of the influenza-specific effector and memory repertoires. Consideration is given to future possible vaccine strategies and to the effects of ageing. It is postulated that understanding the complexities of CD8+ T cell mediated immunity and memory has the potential for improving vaccine design, particularly to combat pandemics caused by newly emerging influenza viruses. In the review various CD8+ T cell epitopes are reviewed, among others SEQ ID NO:1 (ILRGSVAHK), SEQ ID NO:2 (ELRSRYWAI), SEQ ID NO:3 (SRYWAIRTR), SEQ ID NO:4 (CTELKLSDY), SEQ ID NO:5 (GILGFVFTL), SEQ ID NO:6 (SIIPSGPLK), SEQ ID NO:7 (ASCMGLIY), SEQ ID NO:8 (FMYSDFHFI), SEQ ID NO:10 (VSDGGPNLY), SEQ ID NO:12 (NMLSTVLGV), and SEQ ID NO:16 (RRSGAAGAAVK). How such epitopes may be employed to design an appropriate cross protective CTL based vaccine is left undiscussed, beyond the consideration to use a mixture of dominant and subdominant epitopes in a polypeptide-based vaccine as a strategy for conferring cross protective immunity as discussed before by Stoloff & Caparros-Wanderley (2007) and here discussed below under FLU-v.

FLU-v is a synthetic polyepitope polypeptide vaccine composed of conserved T cell epitopes that complement specifically with mouse MHC (H-2Kb) and human HLA (HLA-A*0201) (Stoloff and Caparros-Wanderley 2007). Initially, in an animal study (bearing another HLA system than man), six polypeptides are included in the FLU-v formulation, with polymer (amino acid) length varying from 21 to 35 amino acids. Two of these peptides are derived from influenza M2 and PB1 (comprising SEQ ID NO:12 (NMLSTVLGV) and SEQ ID NO:13 (MMMGMFNML), each without spacer or linker separation), whereas the other four peptides belonged to M1 (comprising SEQ ID NO:5 (GILGFVFTL)) and NP (comprising SEQ ID NO:1 (ILRGSVAHK)) antigens, again without spacer or linker elements. The polypeptides are selected based on the following criteria: (i) length must not be more than 40 amino acids; (ii) composed of at least five human T cell epitopes; and (iii) with a probability of 10⁻¹⁰ or less for any peptide not containing at least one of the identified T cell epitopes. The results of a 2b study in humans (EudraCT: 2016-002134-74) with a lyophilized vaccine composed of four short polypeptides; FLU-5 (32aa), FLU-7 (21aa), FLU-8N (20aa), and FLU-10 (24aa) that originate from conserved regions in internal proteins M1, NPA, NPB, and M2, respectively, however, demonstrate that this polyepitope peptide-based vaccine (in the absence of inducing antibody responses against HA) again falls short in efficacy and can provide only little protection against influenza (Pleguezuelos et al. 2020). Typically, where a one-dose regime showed some significant benefit over placebo-vaccinated subjects, repeated vaccination with the four-polypeptide vaccine did not raise protection, as would be desired of a vaccine in practical use. To the contrary, efficacy of the two-dose regimen is not found statistically significantly different from placebo, making repeat vaccinations with this four-polypeptide epitope prospective vaccine formulation of little use in practice. The authors conclude that the formulation should continue to be evaluated and T-cell immunity explored further as a possible useful correlate of protection against influenza virus infection.

Modified vaccinia Ankara (MVA) is an attenuated vaccinia virus that has been shown to prime T cell responses toward the antigens they carry. Given its promising adjuvant effect, MVA has been used as a vaccine carrier for malaria, human immunodeficiency virus (HIV) and tuberculosis vaccines. MVA offers several advantages as vaccine carrier, such as: (i) excellent safety profile in children and HIV-positive individuals; (ii) great stability; (iii) rapid stimulation of humoral and cellular responses and (iv) various vaccine inoculation routes. Several MVA-based influenza vaccines have been developed and are undergoing rigorous testing. Amongst them, MVA-NP+M1 vaccine (modified vaccinia Ankara expressing virus nucleoprotein and matrix protein 1) has reached the clinical trial phase. The aim of a recent 2b study is to assess whether inducing additional universal responses to conserved CD4 and CD8 T-cell antigens NP and M1 provides added benefit to standard influenza vaccination. However, this vaccine designed to induce T-cell responses to these cross-reactive internal proteins of influenza A did not lead to improved incidence when given within 28 days after standard quadrivalent vaccine immunization (Evans et al. 2022). The trial has been stopped after one season for reasons of futility on the recommendation of the data monitoring committee.

Despite some advances described above, there are no broad-spectrum influenza vaccines available for prevention against flu, illustrating that there is no one-way-street toward obtaining such a desired outcome. Studies in mice hardly improve chances toward that one-way-street scenario, and there exist only few models of transgenic human-HLA expression in mice, thus in vivo studies in transgenic mice to identify immunogenicity mediated by T cells that depend on human MHC I processing activities are limited and will never suffice to provide a full picture of immunogenic activities in humans.

That having been said, there is still a great need for influenza-specific antigenic formulations to develop immunogens and vaccines that lead to efficient induction of broadly protective responses and thus can provide protective immunity and therapeutic activity in the field of influenza control.

### BRIEF SUMMARY

This disclosure discloses a collection of antigenic as well as immunogenic determinant formulations with demonstrated antigenic as well as immunogenic potential and capable of addressing the need for broadly protective responses to provide protective immunity and therapeutic activity in the field of influenza control. Therewith, the disclosure discloses a multi-epitope influenza A vaccine formulation for use in triggering or inducing cross-protective immunity against different influenza strains comprising at least 5 relatively conserved peptide-(amino acid-) sequences each demonstrated capable of eliciting interferon-gamma (IFNγ) secretion by influenza virus specific CD8+-CTL.

In a first embodiment, to accommodate providing the vaccine foremost with conserved antigenic and immunogenic determinant peptides SEQ ID NO:8 (FMYSDFHFI), SEQ ID NO:9 (FVRQCFNPM), SEQ ID NO:12 (NMLSTVLGV), SEQ ID NO:13 (MMMGMFNML), and SEQ ID NO:14 (YSHGTGTGY), derived from currently circulating human IAV strains to accommodate vaccination against those currently circulating human viruses in humans and provide the broad protection desired (see also Table 2 herein), the disclosure discloses a nucleic acid encoding at least 5 of influenza A virus (IAV) derived peptides capable of inducing IFNγ by CD8+ T cells with amino acid sequences SEQ ID NO:8 (FMYSDFHFI), SEQ ID NO:9 (FVRQCFNPM), SEQ ID NO:12 (NMLSTVLGV), SEQ ID NO:13 (MMMGMFNML), and SEQ ID NO:14 (YSHGTGTGY). Such a nucleic acid may be complemented with a nucleic acid encoding at least 5 or 10 of influenza A virus (IAV) derived peptides capable of inducing IFNγ by CD8+ T cells selected from the group of peptides capable of inducing IFNγ by CD8+ T cells with amino acid sequences SEQ ID NO:1 (ILRGSVAHK), SEQ ID NO:2 (ELRSRYWAI), SEQ ID NO:3 (SRYWAIRTR), SEQ ID NO:4 (CTELKLSDY), SEQ ID NO:5 (GILGFVFTL), SEQ ID NO:6 (SIIPSGPLK), SEQ ID NO:7 (ASCMGLIY), SEQ ID NO:10 (VSDGGPNLY), SEQ ID NO:11 (FLKDVMESM), SEQ ID NO:15 (HSNLNDATY), SEQ ID NO:16 (RRSGAAGAAVK), SEQ ID NO:17 (LLTEVETYV), SEQ ID NO:18 (MVLASTTAK), SEQ ID NO:19 (RGINDRNFW), and SEQ ID NO:20 (FLLMDALKL).

To provide for optimal liberation of epitope peptides from polyepitope constructs, irrespective of their order of appearance in the polyepitope polypeptide, the nucleic acid at least encoding SEQ ID NO:8 (FMYSDFHFI), SEQ ID NO:9 (FVRQCFNPM), SEQ ID NO:12 (NMLSTVLGV), SEQ ID NO:13 (MMMGMFNML), and SEQ ID NO:14 (YSHGTGTGY) as disclosed is also provided with genetic information to provide for appropriate spacer (linker) placement adjacent (N- as well as C-terminally) to the peptides (epitopes), as well as with genetic information to provide for a degron, to facilitate correct and optimal cleavage of the peptides from the polyepitope polypeptide according to the disclosure in the proteasome and prevent the creation of neo-epitopes from adjacent epitopes sequences, which can reduce vaccine efficacy (Schubert and Kohlbacher 2016). The here selected epitopes are used to construct an artificial gene encoding the polyepitope polypeptide sequence. Four different constructs were made with or without the genetic information for spacer (less or more supporting the liberation of the epitopes) and with or without the genetic information encoding a proteasome-targeting signal (herein also identified as degron ((Ravid and Hochstrasser 2008)) for docking the polyepitope to the proteasome for optimal antigen processing. Here, the spacer sequence AAY (Ismail, Ahmad, and Azam 2020) was selected to assess the best requirements for optimal liberation of antigenic peptides from polyepitope polypeptide constructs of the disclosure and demonstrated that avoiding dependence on the order of the peptides within the polyepitope polypeptide of the disclosure could be accomplished, which may dictate cleavage probability in the proteasome, to generate a strong increase in the number of correctly cleaved epitopes and a decrease in the neo-immunogenicity of the complete construct and have therewith independent recovery of individual epitopes and subsequent MHC I processing irrespective of the order in which they are located in the polyepitope construct. The nucleic acid at least encoding SEQ ID NO:8 (FMYSDFHFI), SEQ ID NO:9 (FVRQCFNPM), SEQ ID NO:12 (NMLSTVLGV), SEQ ID NO:13 (MMMGMFNML), and SEQ ID NO:14 (YSHGTGTGY) as disclosed is additionally provided with the genetic information encoding a proteasome-targeting signal (herein also identified as degron ((Ravid and Hochstrasser 2008))) to provide appropriate targeting of the polyepitope polypeptide transcribable from the nucleic acid to the proteasome and further improved cleavage probability therein. As discussed in detail by Ravid and Hochstrasser, most short-lived proteins are distinguished by localized structure determinants (‘signals’) that target them to the ubiquitin ligase machinery or to the proteasome (or lysosome in some cases). A degradation signal or ‘degron’, is usually defined as a minimal element within a protein that is sufficient for recognition and degradation by a proteolytic apparatus. An important property of degrons is that they are transferable. That is, genetically engineered attachment of such sequences confers metabolic instability (a shorter half-life) on otherwise longer-lived proteins. To provide for such a degron, here ubiquitin was selected, preferably placed N-terminally to the polyepitope polypeptide construct. Such a nucleic acid as disclosed herein may comprise DNA or RNA or both and may provide an antigenic as well as an immunogenic determinant formulation by allowing expression of the individual peptides with SEQ ID NO:8 (FMYSDFHFI), SEQ ID NO:9 (FVRQCFNPM), SEQ ID NO:12 (NMLSTVLGV), SEQ ID NO:13 (MMMGMFNML), and SEQ ID NO:14 (YSHGTGTGY) in a cell or vaccinated subject and mounting an immune response. In a further embodiment, the disclosure discloses the nucleic acid according to the disclosure additionally encoding another 5 of influenza A virus (IAV) derived peptides encoding epitopes capable of inducing IFNγ by CD8+ T cells, the another 5 peptides encoding epitopes having SEQ ID NO:5 (GILGFVFTL), SEQ ID NO:3 (SRYWAIRTR), SEQ ID NO:16 (RRSGAAGAAVK), SEQ ID NO:2 (ELRSRYWAI), and SEQ ID NO:4 (CTELKLSDY).

In a further embodiment, the disclosure discloses the nucleic acid according to the disclosure encoding at least 15, preferably at least 18 of influenza A virus (IAV) derived peptides encoding epitopes capable of inducing IFNγ by CD8+ T cells, selected from the group of peptides capable of inducing IFNγ by CD8+ T cells with amino acid sequences SEQ ID NO:1 (ILRGSVAHK), SEQ ID NO:2 (ELRSRYWAI), SEQ ID NO:3 (SRYWAIRTR), SEQ ID NO:4 (CTELKLSDY), SEQ ID NO:5 (GILGFVFTL), SEQ ID NO:6 (SIIPSGPLK), SEQ ID NO:7 (ASCMGLIY), SEQ ID NO:8 (FMYSDFHFI), SEQ ID NO:9 (FVRQCFNPM), SEQ ID NO:10 (VSDGGPNLY), SEQ ID NO:11 (FLKDVMESM), SEQ ID NO:12 (NMLSTVLGV), SEQ ID NO:13 (MMMGMFNML), SEQ ID NO:14 (YSHGTGTGY), SEQ ID NO:15 (HSNLNDATY), SEQ ID NO:16 (RRSGAAGAAVK), SEQ ID NO:17 (LLTEVETYV), SEQ ID NO:18 (MVLASTTAK), SEQ ID NO:19 (RGINDRNFW) and SEQ ID NO:20 (FLLMDALKL).

In a further embodiment, the disclosure discloses the nucleic acid according to the disclosure encoding 20 of influenza A virus (IAV) derived peptides encoding epitopes capable of inducing IFNγ by CD8+ T cells, with amino acid sequences SEQ ID NO:1 (ILRGSVAHK), SEQ ID NO:2 (ELRSRYWAI), SEQ ID NO:3 (SRYWAIRTR), SEQ ID NO:4 (CTELKLSDY), SEQ ID NO:5 (GILGFVFTL), SEQ ID NO:6 (SIIPSGPLK), SEQ ID NO:7 (ASCMGLIY), SEQ ID NO:8 (FMYSDFHFI), SEQ ID NO:9 (FVRQCFNPM), SEQ ID NO:10 (VSDGGPNLY), SEQ ID NO:11 (FLKDVMESM), SEQ ID NO:12 (NMLSTVLGV), SEQ ID NO:13 (MMMGMFNML), SEQ ID NO:14 (YSHGTGTGY), SEQ ID NO:15 (HSNLNDATY), SEQ ID NO:16 (RRSGAAGAAVK), SEQ ID NO:17 (LLTEVETYV), SEQ ID NO:18 (MVLASTTAK), SEQ ID NO:19 (RGINDRNFW) and SEQ ID NO:20 (FLLMDALKL).

Additionally disclosed is a nucleic acid (polynucleotide) at least encoding 6 of influenza A virus (IAV) derived peptides encoding epitopes capable of inducing IFNγ by CD8+ T cells, the at least 6 peptides encoding epitopes having SEQ ID NO:4 (CTELKLSDY), SEQ ID NO:8 (FMYSDFHFI), SEQ ID NO:9 (FVRQCFNPM), SEQ ID NO:12 (NMLSTVLGV), SEQ ID NO:13 (MMMGMFNML), and SEQ ID NO:14 (YSHGTGTGY) and as disclosed is additionally provided with the genetic information encoding a proteasome-targeting signal (herein also identified as degron ((Ravid and Hochstrasser 2008))) to provide appropriate targeting of the polyepitope polypeptide transcribable from the nucleic acid to the proteasome and further improved cleavage probability therein. A nucleic acid as disclosed herein may comprise DNA or RNA or both and may provide an antigenic as well as an immunogenic determinant formulation by allowing expression of the individual peptides.

Additionally disclosed is a nucleic acid at least encoding 6 of influenza A virus (IAV) derived peptides encoding epitopes capable of inducing IFNγ by CD8+ T cells, the at least 6 peptides encoding epitopes having SEQ ID NO:5 (GILGFVFTL), SEQ ID NO:8 (FMYSDFHFI), SEQ ID NO:9 (FVRQCFNPM), SEQ ID NO:12 (NMLSTVLGV), SEQ ID NO:13 (MMMGMFNML), and SEQ ID NO:14 (YSHGTGTGY), and as disclosed is additionally provided with the genetic information encoding appropriate spaces and a degron. Such a nucleic acid as disclosed herein may comprise DNA or RNA or both and may provide an antigenic as well as an immunogenic determinant formulation by allowing expression of the individual peptides.

Additionally disclosed is a nucleic acid at least encoding 6 of influenza A virus (IAV) derived peptides encoding epitopes capable of inducing IFNγ by CD8+ T cells, the at least 6 peptides encoding epitopes having SEQ ID NO:8 (FMYSDFHFI), SEQ ID NO:9 (FVRQCFNPM), SEQ ID NO:10 (VSDGGPNLY), SEQ ID NO:12 (NMLSTVLGV), SEQ ID NO:13 (MMMGMFNML), and SEQ ID NO:14 (YSHGTGTGY), and as disclosed is additionally provided with the genetic information encoding appropriate spaces and a degron. Such a nucleic acid as disclosed herein may comprise DNA or RNA or both and may provide an antigenic as well as an immunogenic determinant formulation by allowing expression of the individual peptides.

Additionally disclosed is a nucleic acid at least encoding 7 of influenza A virus (IAV) derived peptides encoding epitopes capable of inducing IFNγ by CD8+ T cells, the at least 7 peptides encoding epitopes having SEQ ID NO:4 (CTELKLSDY), SEQ ID NO:5 (GILGFVFTL), SEQ ID NO:8 (FMYSDFHFI), SEQ ID NO:9 (FVRQCFNPM), SEQ ID NO:12 (NMLSTVLGV), SEQ ID NO:13 (MMMGMFNML), and SEQ ID NO:14 (YSHGTGTGY), and as disclosed is additionally provided with the genetic information encoding appropriate spaces and a degron. A nucleic acid as disclosed herein may comprise DNA or RNA or both and may provide an antigenic as well as an immunogenic determinant formulation by allowing expression of the individual peptides.

Additionally disclosed is a nucleic acid at least encoding 7 of influenza A virus (IAV) derived peptides encoding epitopes capable of inducing IFNγ by CD8+ T cells, the at least 7 peptides encoding epitopes having SEQ ID NO:4 (CTELKLSDY), SEQ ID NO:10 (VSDGGPNLY), SEQ ID NO:8 (FMYSDFHFI), SEQ ID NO:9 (FVRQCFNPM), SEQ ID NO:12 (NMLSTVLGV), SEQ ID NO:13 (MMMGMFNML), and SEQ ID NO:14 (YSHGTGTGY), and as disclosed is additionally provided with the genetic information encoding appropriate spaces and a degron. A nucleic acid as disclosed herein may comprise DNA or RNA or both and may provide an antigenic as well as an immunogenic determinant formulation by allowing expression of the individual peptides.

Additionally disclosed is a nucleic acid at least encoding 7 of influenza A virus (IAV) derived peptides encoding epitopes capable of inducing IFNγ by CD8+ T cells, the at least 7 peptides encoding epitopes having SEQ ID NO:5 (GILGFVFTL), SEQ ID NO:8 (FMYSDFHFI), SEQ ID NO:9 (FVRQCFNPM), SEQ ID NO:10 (VSDGGPNLY), SEQ ID NO:12 (NMLSTVLGV), SEQ ID NO:13 (MMMGMFNML), and SEQ ID NO:14 (YSHGTGTGY), and as disclosed is additionally provided with the genetic information encoding appropriate spaces and a degron. A nucleic acid as disclosed herein may comprise DNA or RNA or both and may provide an antigenic as well as an immunogenic determinant formulation by allowing expression of the individual peptides.

Additionally disclosed is a nucleic acid at least encoding 8 of influenza A virus (IAV) derived peptides encoding epitopes capable of inducing IFNγ by CD8+ T cells, the at least 8 peptides encoding epitopes having SEQ ID NO:4 (CTELKLSDY), SEQ ID NO:5 (GILGFVFTL), SEQ ID NO:8 (FMYSDFHFI), SEQ ID NO:9 (FVRQCFNPM), SEQ ID NO:10 (VSDGGPNLY), SEQ ID NO:12 (NMLSTVLGV), SEQ ID NO:13 (MMMGMFNML), and SEQ ID NO:14 (YSHGTGTGY), and as disclosed is additionally provided with the genetic information encoding appropriate spaces and a degron. A nucleic acid as disclosed herein may comprise DNA or RNA or both and may provide an antigenic as well as an immunogenic determinant formulation by allowing expression of the individual peptides.

Additionally disclosed is a nucleic acid at least encoding another 6 of influenza A virus (IAV) derived peptides encoding epitopes capable of inducing IFNγ by CD8+ T cells, the another 6 peptides encoding epitopes having SEQ ID NO:5 (GILGFVFTL), SEQ ID NO:3 (SRYWAIRTR)), SEQ ID NO:10 (VSDGGPNLY), SEQ ID NO:16 (RRSGAAGAAVK), SEQ ID NO:2 (ELRSRYWAI), and SEQ ID NO:4 (CTELKLSDY), and as disclosed is additionally provided with the genetic information encoding appropriate spaces and a degron. A nucleic acid as disclosed herein may comprise DNA or RNA or both and may provide an antigenic as well as an immunogenic determinant formulation by allowing expression of the individual peptides. In a further embodiment, the disclosure discloses the RNA or DNA nucleic acid according to the disclosure wherein the peptide spacer comprises tripeptide AAY.

In a further embodiment, disclosed is RNA or DNA nucleic acid according to the disclosure wherein the degron comprises ubiquitin. In a further embodiment, the disclosure discloses the nucleic acid vector, virus, cell, or formulation comprising the RNA or DNA nucleic acid according to the disclosure. In a further embodiment, the disclosure discloses a proteinaceous formulation comprising a polyepitope polypeptide derived from the nucleic acid, vector, virus, or cell according to the disclosure.

In a further embodiment, disclosed is a synthetic polyepitope polypeptide and/or antigenic and immunogenic determinant formulation comprising at least 5 of influenza A virus (IAV) derived peptides encoding epitopes capable of inducing IFNγ by CD8+ T cells, the at least 5 peptides encoding epitopes having SEQ ID NO:8 (FMYSDFHFI), SEQ ID NO:9 (FVRQCFNPM), SEQ ID NO:12 (NMLSTVLGV), SEQ ID NO:13 (MMMGMFNML), and SEQ ID NO:14 (YSHGTGTGY), the polypeptide also provided with an appropriate peptide spacer (linker) placement adjacent to peptides encoding epitopes and provided with a degron. In a further embodiment, the disclosure discloses a synthetic polyepitope polypeptide and/or antigenic and immunogenic determinant formulation comprising at least 6 of influenza A virus (IAV) derived peptides encoding epitopes capable of inducing IFNγ by CD8+ T cells, the at least 6 peptides encoding epitopes having SEQ ID NO:4 (CTELKLSDY), SEQ ID NO:8 (FMYSDFHFI), SEQ ID NO:9 (FVRQCFNPM), SEQ ID NO:12 (NMLSTVLGV), SEQ ID NO:13 (MMMGMFNML), and SEQ ID NO:14 (YSHGTGTGY), the polypeptide also provided with an appropriate peptide spacer (linker) placement adjacent to peptides encoding epitopes and provided with a degron.

In a further embodiment, disclosed is a synthetic polyepitope polypeptide and/or antigenic and immunogenic determinant formulation comprising at least 6 of influenza A virus (IAV) derived peptides encoding epitopes capable of inducing IFNγ by CD8+ T cells, the at least 6 peptides encoding epitopes having SEQ ID NO:5 (GILGFVFTL), SEQ ID NO:8 (FMYSDFHFI), SEQ ID NO:9 (FVRQCFNPM), SEQ ID NO:12 (NMLSTVLGV), SEQ ID NO:13 (MMMGMFNML), and SEQ ID NO:14 (YSHGTGTGY), the polypeptide also provided with an appropriate peptide spacer (linker) placement adjacent to peptides encoding epitopes and provided with a degron.

In a further embodiment, disclosed is a synthetic polyepitope polypeptide and/or antigenic and immunogenic determinant formulation comprising at least 6 of influenza A virus (IAV) derived peptides encoding epitopes capable of inducing IFNγ by CD8+ T cells, the at least 6 peptides encoding epitopes having SEQ ID NO:8 (FMYSDFHFI), SEQ ID NO:9 (FVRQCFNPM), SEQ ID NO:10 (VSDGGPNLY), SEQ ID NO:12 (NMLSTVLGV), SEQ ID NO:13 (MMMGMFNML), and SEQ ID NO:14 (YSHGTGTGY), the polypeptide also provided with an appropriate peptide spacer (linker) placement adjacent to peptides encoding epitopes and provided with a degron.

In a further embodiment, disclosed is a synthetic polyepitope polypeptide and/or antigenic and immunogenic determinant formulation comprising at least 7 of influenza A virus (IAV) derived peptides encoding epitopes capable of inducing IFNγ by CD8+ T cells, the at least 7 peptides encoding epitopes having SEQ ID NO:4 (CTELKLSDY), SEQ ID NO:5 (GILGFVFTL), SEQ ID NO:8 (FMYSDFHFI), SEQ ID NO:9 (FVRQCFNPM), SEQ ID NO:12 (NMLSTVLGV), SEQ ID NO:13 (MMMGMFNML), and SEQ ID NO:14 (YSHGTGTGY), the polypeptide also provided with an appropriate peptide spacer (linker) placement adjacent to peptides encoding epitopes and provided with a degron.

In a further embodiment, disclosed is a synthetic polyepitope polypeptide and/or antigenic and immunogenic determinant formulation comprising at least 7 of influenza A virus (IAV) derived peptides encoding epitopes capable of inducing IFNγ by CD8+ T cells, the at least 7 peptides encoding epitopes having SEQ ID NO:4 (CTELKLSDY), SEQ ID NO:10 (VSDGGPNLY), SEQ ID NO:8 (FMYSDFHFI), SEQ ID NO:9 (FVRQCFNPM), SEQ ID NO:12 (NMLSTVLGV), SEQ ID NO:13 (MMMGMFNML), and SEQ ID NO:14 (YSHGTGTGY), the polypeptide also provided with an appropriate peptide spacer (linker) placement adjacent to peptides encoding epitopes and provided with a degron.

In a further embodiment, disclosed is a synthetic polyepitope polypeptide and/or antigenic and immunogenic determinant formulation comprising at least 7 of influenza A virus (IAV) derived peptides encoding epitopes capable of inducing IFNγ by CD8+ T cells, the at least 7 peptides encoding epitopes having SEQ ID NO:5 (GILGFVFTL), SEQ ID NO:8 (FMYSDFHFI), SEQ ID NO:9 (FVRQCFNPM), SEQ ID NO:10 (VSDGGPNLY), SEQ ID NO:12 (NMLSTVLGV), SEQ ID NO:13 (MMMGMFNML), and SEQ ID NO:14 (YSHGTGTGY), the polypeptide also provided with an appropriate peptide spacer (linker) placement adjacent to peptides encoding epitopes and provided with a degron.

In a further embodiment, disclosed is a synthetic polyepitope polypeptide and/or antigenic and immunogenic determinant formulation comprising at least 8 of influenza A virus (IAV) derived peptides encoding epitopes capable of inducing IFNγ by CD8+ T cells, the at least 8 peptides encoding epitopes having SEQ ID NO:4 (CTELKLSDY), SEQ ID NO:5 (GILGFVFTL), SEQ ID NO:8 (FMYSDFHFI), SEQ ID NO:9 (FVRQCFNPM), SEQ ID NO:10 (VSDGGPNLY), SEQ ID NO:12 (NMLSTVLGV), SEQ ID NO:13 (MMMGMFNML), and SEQ ID NO:14 (YSHGTGTGY), the polypeptide also provided with an appropriate peptide spacer (linker) placement adjacent to peptides encoding epitopes and provided with a degron.

In a further embodiment, disclosed is a synthetic polyepitope polypeptide and/or antigenic and immunogenic determinant formulation to the disclosure additionally provided with another 5 of influenza A virus (IAV) derived peptides encoding epitopes capable of inducing IFNγ by CD8+ T cells, the another 5 peptides encoding epitopes having SEQ ID NO:5 (GILGFVFTL), SEQ ID NO:3 (SRYWAIRTR), SEQ ID NO:16 (RRSGAAGAAVK), SEQ ID NO:2 (ELRSRYWAI), and SEQ ID NO:4 (CTELKLSDY), the polypeptide also provided with an appropriate peptide spacer (linker) placement adjacent to peptides encoding epitopes and provided with a degron.

In a further embodiment, disclosed is a synthetic polyepitope polypeptide and/or antigenic and immunogenic determinant formulation to the disclosure additionally provided with another 6 of influenza A virus (IAV) derived peptides encoding epitopes capable of inducing IFNγ by CD8+ T cells, the another 6 peptides encoding epitopes having SEQ ID NO:5 (GILGFVFTL), SEQ ID NO:3 (SRYWAIRTR)), SEQ ID NO:10 (VSDGGPNLY), SEQ ID NO:16 (RRSGAAGAAVK), SEQ ID NO:2 (ELRSRYWAI), and SEQ ID NO:4 (CTELKLSDY), the polypeptide also provided with an appropriate peptide spacer (linker) placement adjacent to peptides encoding epitopes and provided with a degron.

In a further embodiment, disclosed is a synthetic polyepitope polypeptide and/or antigenic and immunogenic determinant formulation according to the disclosure comprising at least 15, preferably at least 18 of influenza A virus (IAV) derived peptides capable of inducing IFNγ by CD8+ T cells selected from the group of peptides capable of inducing IFNγ by CD8+ T cells with amino acid sequences SEQ ID NO:1 (ILRGSVAHK), SEQ ID NO:2 (ELRSRYWAI), SEQ ID NO:3 (SRYWAIRTR), SEQ ID NO:4 (CTELKLSDY), SEQ ID NO:5 (GILGFVFTL), SEQ ID NO:6 (SIIPSGPLK), SEQ ID NO:7 (ASCMGLIY), SEQ ID NO:8 (FMYSDFHFI), SEQ ID NO:9 (FVRQCFNPM), SEQ ID NO:10 (VSDGGPNLY), SEQ ID NO:11 (FLKDVMESM), SEQ ID NO:12 (NMLSTVLGV), SEQ ID NO:13 (MMMGMFNML), SEQ ID NO:14 (YSHGTGTGY), SEQ ID NO:15 (HSNLNDATY), SEQ ID NO:16 (RRSGAAGAAVK), SEQ ID NO:17 (LLTEVETYV), SEQ ID NO:18 (MVLASTTAK), SEQ ID NO:19 (RGINDRNFW) and SEQ ID NO:20 (FLLMDALKL).

In a further embodiment, disclosed is a synthetic polyepitope polypeptide and/or antigenic and immunogenic determinant formulation according to the disclosure comprising 20 of influenza A virus (IAV) derived peptides encoding epitopes capable of inducing IFNγ by CD8+ T cells, with amino acid sequences SEQ ID NO:1 (ILRGSVAHK), SEQ ID NO:2 (ELRSRYWAI), SEQ ID NO:3 (SRYWAIRTR), SEQ ID NO:4 (CTELKLSDY), SEQ ID NO:5 (GILGFVFTL), SEQ ID NO:6 (SIIPSGPLK), SEQ ID NO:7 (ASCMGLIY), SEQ ID NO:8 (FMYSDFHFI), SEQ ID NO:9 (FVRQCFNPM), SEQ ID NO:10 (VSDGGPNLY), SEQ ID NO:11 (FLKDVMESM), SEQ ID NO:12 (NMLSTVLGV), SEQ ID NO:13 (MMMGMFNML), SEQ ID NO:14 (YSHGTGTGY), SEQ ID NO:15 (HSNLNDATY), SEQ ID NO:16 (RRSGAAGAAVK), SEQ ID NO:17 (LLTEVETYV), SEQ ID NO:18 (MVLASTTAK), SEQ ID NO:19 (RGINDRNFW) and SEQ ID NO:20 (FLLMDALKL).

In a further embodiment, disclosed is an antigenic and immunogenic determinant formulation comprising the nucleic acid according to the disclosure or comprising the vector, virus, cell, or formulation according to the disclosure or comprising a proteinaceous formulation according to the disclosure or a synthetic polyepitope polypeptide according to the disclosure.

In a further embodiment, disclosed is an immunogenic formulation comprising a nucleic acid formulation according to the disclosure or a proteinaceous formulation according to the disclosure or a synthetic polyepitope polypeptide according to the disclosure. In a further embodiment, the disclosure discloses a vaccine formulation obtainable by mixing an antigenic and immunogenic determinant formulation according to the disclosure and/or an immunogenic formulation according to the disclosure with a pharmaceutically acceptable excipient.

In a further embodiment, disclosed is a vaccine formulation according to the disclosure for use in vaccination together with or in addition to or preferably concomitant with, preferably yearly, vaccination with a vaccine directed at generating humoral vaccine responses toward an influenza virus hemagglutinin protein.

In a further embodiment, disclosed is a nucleic acid encoding at least 5 of the above influenza A virus (IAV) derived peptides capable of inducing IFNγ by CD8+ T cells selected from the group of peptides capable of inducing IFNγ by CD8+ T cells with amino acid sequences SEQ ID NO:1 (ILRGSVAHK), SEQ ID NO:2 (ELRSRYWAI), SEQ ID NO:3 (SRYWAIRTR), SEQ ID NO:4 (CTELKLSDY), SEQ ID NO:5 (GILGFVFTL), SEQ ID NO:6 (SIIPSGPLK), SEQ ID NO:7 (ASCMGLIY), SEQ ID NO:8 (FMYSDFHFI), SEQ ID NO:9 (FVRQCFNPM), SEQ ID NO:10 (VSDGGPNLY), SEQ ID NO:11 (FLKDVMESM), SEQ ID NO:12 (NMLSTVLGV), SEQ ID NO:13 (MMMGMFNML), SEQ ID NO:14 (YSHGTGTGY), SEQ ID NO:15 (HSNLNDATY), SEQ ID NO:16 (RRSGAAGAAVK), SEQ ID NO:17 (LLTEVETYV), SEQ ID NO:18 (MVLASTTAK), SEQ ID NO:19 (RGINDRNFW) and SEQ ID NO:20 (FLLMDALKL) (see also Table 1 herein). Such a nucleic acid as provided herein may comprise DNA or RNA or both and may function as an antigenic as well as immunogenic determinant formulation by allowing expression of the peptides in a cell or vaccinated subject and mounting an immune response.

In a preferred embodiment, to accommodate providing the vaccine foremost with conserved antigenic and immunogenic determinant peptides SEQ ID NO:8 (FMYSDFHFI), SEQ ID NO:9 (FVRQCFNPM), SEQ ID NO:12 (NMLSTVLGV), SEQ ID NO:13 (MMMGMFNML), and SEQ ID NO:14 (YSHGTGTGY), derived from currently circulating human IAV strains to accommodate vaccination against those currently circulating human viruses in humans and provide the broad protection desired (see also Table 2 herein), the disclosure discloses a nucleic acid encoding at least 5 of influenza A virus (IAV) derived peptides capable of inducing IFNγ by CD8+ T cells with amino acid sequences SEQ ID NO:8 (FMYSDFHFI), SEQ ID NO:9 (FVRQCFNPM), SEQ ID NO:12 (NMLSTVLGV), SEQ ID NO:13 (MMMGMFNML), and SEQ ID NO:14 (YSHGTGTGY). Such a nucleic acid may be complemented with a nucleic acid encoding at least 5 or 10 of influenza A virus (IAV) derived peptides capable of inducing IFNγ by CD8+ T cells selected from the group of peptides capable of inducing IFNγ by CD8+ T cells with amino acid sequences SEQ ID NO:1 (ILRGSVAHK), SEQ ID NO:2 (ELRSRYWAI), SEQ ID NO:3 (SRYWAIRTR), SEQ ID NO:4 (CTELKLSDY), SEQ ID NO:5 (GILGFVFTL), SEQ ID NO:6 (SIIPSGPLK), SEQ ID NO:7 (ASCMGLIY), SEQ ID NO:10 (VSDGGPNLY), SEQ ID NO:11 (FLKDVMESM), SEQ ID NO:15 (HSNLNDATY), SEQ ID NO:16 (RRSGAAGAAVK), SEQ ID NO:17 (LLTEVETYV), SEQ ID NO:18 (MVLASTTAK), SEQ ID NO:19 (RGINDRNFW), and SEQ ID NO:20 (FLLMDALKL).

In a further preferred embodiment, disclosed is complementing the nucleic acid as provided herein with at least the nucleic acid of 5 of influenza A virus (IAV) derived peptides capable of inducing IFNγ by CD8+ T cells selected from the group of peptides capable of inducing IFNγ by CD8+ T cells with immunodominant amino acid sequences SEQ ID NO:5 (GILGFVFTL), SEQ ID NO:3 (SRYWAIRTR), SEQ ID NO:16 (RRSGAAGAAVK), SEQ ID NO:2 (ELRSRYWAI), and SEQ ID NO:4 (CTELKLSDY) (see also table 5). In another further preferred embodiment, the disclosure discloses to complement the nucleic acid as provided herein with at least the nucleic acid of 5 of influenza A virus (IAV) derived peptides capable of inducing IFNγ by CD8+ T cells selected from the group of peptides capable of inducing IFNγ by CD8+ T cells with immunodominant amino acid sequences SEQ ID NO:5 (GILGFVFTL), SEQ ID NO:10 (VSDGGPNLY), SEQ ID NO:1 (ILRGSVAHK), SEQ ID NO:2 (ELRSRYWAI), and SEQ ID NO:3 (SRYWAIRTR)(see also table 6) to provide a vaccine with further improved HLA coverage. In yet another further preferred embodiment, the disclosure discloses to complement the nucleic acid as provided herein with at least the nucleic acid of 5 of influenza A virus (IAV) derived peptides capable of inducing IFNγ by CD8+ T cells selected from the group of peptides capable of inducing IFNγ by CD8+ T cells with amino acid sequences SEQ ID NO:5 (GILGFVFTL), SEQ ID NO:10 (VSDGGPNLY), SEQ ID NO:20 (FLLMDALKL), SEQ ID NO:2 (ELRSRYWAI), SEQ ID NO:3 (SRYWAIRTR) (see also table 7) to provide a vaccine response directed at a broad group of viral proteins. Such a nucleic acid as provided herein may comprise DNA or RNA or both and may function as an antigenic and immunogenic determinant formulation by allowing expression of the peptides in a cell or vaccinated subject.

In another preferred embodiment, to, for example, accommodate providing the ultimate vaccine foremost with conserved antigenic and immunogenic determinant peptides SEQ ID NO:8 (FMYSDFHFI), SEQ ID NO:12 (NMLSTVLGV), SEQ ID NO:13 (MMMGMFNML), SEQ ID NO:14 (YSHGTGTGY), and SEQ ID NO:15 (HSNLNDATY), derived from currently circulating swine IAV strains to accommodate vaccination against those currently circulating swine viruses in swine and provide broad protection (see also Table 3 herein), the disclosure discloses a nucleic acid encoding at least 5 of influenza A virus (IAV) derived peptides capable of inducing IFNγ by CD8+ T cells with amino acid SEQ ID NO:8 (FMYSDFHFI), SEQ ID NO:12 (NMLSTVLGV), SEQ ID NO:13 (MMMGMFNML), SEQ ID NO:14 (YSHGTGTGY), and SEQ ID NO:15 (HSNLNDATY). Such a nucleic acid may be complemented with a nucleic acid encoding at least 5 or 10 of influenza A virus (IAV) derived peptides capable of inducing IFNγ by CD8+ T cells selected from the group of peptides capable of inducing IFNγ by CD8+ T cells with amino acid sequences SEQ ID NO:1 (ILRGSVAHK), SEQ ID NO:2 (ELRSRYWAI), SEQ ID NO:3 (SRYWAIRTR), SEQ ID NO:4 (CTELKLSDY), SEQ ID NO:5 (GILGFVFTL), SEQ ID NO:6 (SIIPSGPLK), SEQ ID NO:7 (ASCMGLIY), SEQ ID NO:9 (FVRQCFNPM), SEQ ID NO:10 (VSDGGPNLY), SEQ ID NO:11 (FLKDVMESM), SEQ ID NO:16 (RRSGAAGAAVK), SEQ ID NO:17 (LLTEVETYV), SEQ ID NO:18 (MVLASTTAK), SEQ ID NO:19 (RGINDRNFW), and SEQ ID NO:20 (FLLMDALKL). In a further preferred embodiment, the disclosure discloses to complement the nucleic acid as provided herein with at least the nucleic acid of 5 of influenza A virus (IAV) derived peptides capable of inducing IFNγ by CD8+ T cells selected from the group of peptides capable of inducing IFNγ by CD8+ T cells with immunodominant amino acid sequences SEQ ID NO:5 (GILGFVFTL), SEQ ID NO:3 (SRYWAIRTR), SEQ ID NO:16 (RRSGAAGAAVK), SEQ ID NO:2 (ELRSRYWAI), and SEQ ID NO:4 (CTELKLSDY) (see also table 5). In another further preferred embodiment, the disclosure discloses to complement the nucleic acid as provided herein with at least the nucleic acid of 5 of influenza A virus (IAV) derived peptides capable of inducing IFNγ by CD8+ T cells selected from the group of peptides capable of inducing IFNγ by CD8+ T cells with immunodominant amino acid sequences SEQ ID NO:5 (GILGFVFTL), SEQ ID NO:10 (VSDGGPNLY), SEQ ID NO:1 (ILRGSVAHK), SEQ ID NO:2 (ELRSRYWAI), and SEQ ID NO:3 (SRYWAIRTR)(see also table 6) to provide a vaccine with further improved HLA coverage. In yet another further preferred embodiment, the disclosure discloses to complement the nucleic acid as provided herein with at least the nucleic acid of 5 of influenza A virus (IAV) derived peptides capable of inducing IFNγ by CD8+ T cells selected from the group of peptides capable of inducing IFNγ by CD8+ T cells with amino acid sequences SEQ ID NO:5 (GILGFVFTL), SEQ ID NO:10 (VSDGGPNLY), SEQ ID NO:20 (FLLMDALKL), SEQ ID NO:2 (ELRSRYWAI), SEQ ID NO:3 (SRYWAIRTR) (see also table 7) to provide a vaccine response directed at a broad group of viral proteins. Such a nucleic acid as provided herein may comprise DNA or RNA or both and may function as an antigenic and immunogenic determinant formulation by allowing expression of the peptides in a cell or vaccinated subject.

In another preferred embodiment, disclosed is a nucleic acid encoding at least 6 of influenza A virus (IAV) derived peptides capable of inducing IFNγ by CD8+ T cells with amino acid sequences SEQ ID NO:8 (FMYSDFHFI), SEQ ID NO:9 (FVRQCFNPM), SEQ ID NO:12 (NMLSTVLGV), SEQ ID NO:13 (MMMGMFNML), SEQ ID NO:14 (YSHGTGTGY), and SEQ ID NO:15 (HSNLNDATY) and derived from currently circulating human and swine IAV strains to accommodate vaccination against possibly circulating swine influenza A viruses in humans and provide the additional protection desired (see also Tables 2 and 3 herein). In a further preferred embodiment, the disclosure discloses to complement the nucleic acid as provided herein with at least the nucleic acid of 5 of influenza A virus (IAV) derived peptides capable of inducing IFNγ by CD8+ T cells selected from the group of peptides capable of inducing IFNγ by CD8+ T cells with immunodominant amino acid sequences SEQ ID NO:5 (GILGFVFTL), SEQ ID NO:3 (SRYWAIRTR), SEQ ID NO:16 (RRSGAAGAAVK), SEQ ID NO:2 (ELRSRYWAI), and SEQ ID NO:4 (CTELKLSDY) (see also table 5). Such a nucleic acid as provided herein may comprise DNA or RNA or both and may function as an antigenic and immunogenic determinant formulation by allowing expression of the peptides in a cell or vaccinated subject.

In another preferred embodiment, to, for example, accommodate providing the vaccine foremost with conserved antigenic and immunogenic determinant peptides SEQ ID NO:8 (FMYSDFHFI), SEQ ID NO:14 (YSHGTGTGY), SEQ ID NO:13 (MMMGMFNML), SEQ ID NO:12 (NMLSTVLGV), and SEQ ID NO:19 (RGINDRNFW), derived from currently circulating avian IAV strains to accommodate vaccination against those currently circulating avian viruses in avian and provide broad protection (see also Table 4 herein), the disclosure discloses a nucleic acid encoding at least 5 of influenza A virus (IAV) derived peptides capable of inducing IFNγ by CD8+ T cells with amino acid SEQ ID NO:8 (FMYSDFHFI), SEQ ID NO:14 (YSHGTGTGY), SEQ ID NO:13 (MMMGMFNML), SEQ ID NO:12 (NMLSTVLGV), and SEQ ID NO:19 (RGINDRNFW). Such a nucleic acid may be complemented with a nucleic acid encoding at least 5 or 10 of influenza A virus (IAV) derived peptides capable of inducing IFNγ by CD8+ T cells selected from the group of peptides capable of inducing IFNγ by CD8+ T cells with amino acid sequences SEQ ID NO:1 (ILRGSVAHK), SEQ ID NO:2 (ELRSRYWAI), SEQ ID NO:3 (SRYWAIRTR), SEQ ID NO:4 (CTELKLSDY), SEQ ID NO:5 (GILGFVFTL), SEQ ID NO:6 (SIIPSGPLK), SEQ ID NO:7 (ASCMGLIY), SEQ ID NO:9 (FVRQCFNPM), SEQ ID NO:10 (VSDGGPNLY), SEQ ID NO:11 (FLKDVMESM), SEQ ID NO:15 (HSNLNDATY), SEQ ID NO:16 (RRSGAAGAAVK), SEQ ID NO:17 (LLTEVETYV), SEQ ID NO:18 (MVLASTTAK), and SEQ ID NO:20 (FLLMDALKL). In a further preferred embodiment, the disclosure discloses to complement the nucleic acid as provided herein with at least the nucleic acid of 5 of influenza A virus (IAV) derived peptides capable of inducing IFNγ by CD8+ T cells selected from the group of peptides capable of inducing IFNγ by CD8+ T cells with immunodominant amino acid sequences SEQ ID NO:5 (GILGFVFTL), SEQ ID NO:3 (SRYWAIRTR), SEQ ID NO:16 (RRSGAAGAAVK), SEQ ID NO:2 (ELRSRYWAI), and SEQ ID NO:4 (CTELKLSDY)(see also table 5). In another further preferred embodiment, the disclosure discloses to complement the nucleic acid as provided herein with at least the nucleic acid of 5 of influenza A virus (IAV) derived peptides capable of inducing IFNγ by CD8+ T cells selected from the group of peptides capable of inducing IFNγ by CD8+ T cells with immunodominant amino acid sequences SEQ ID NO:5 (GILGFVFTL), SEQ ID NO:10 (VSDGGPNLY), SEQ ID NO:1 (ILRGSVAHK), SEQ ID NO:2 (ELRSRYWAI), and SEQ ID NO:3 (SRYWAIRTR) (see also table 6) to provide a vaccine with further improved HLA coverage. In yet another further preferred embodiment, the disclosure discloses to complement the nucleic acid as provided herein with at least the nucleic acid of 5 of influenza A virus (IAV) derived peptides capable of inducing IFNγ by CD8+ T cells selected from the group of peptides capable of inducing IFNγ by CD8+ T cells with amino acid sequences SEQ ID NO:5 (GILGFVFTL), SEQ ID NO:10 (VSDGGPNLY), SEQ ID NO:20 (FLLMDALKL), SEQ ID NO:2 (ELRSRYWAI), SEQ ID NO:3 (SRYWAIRTR) (see also table 7) to provide a vaccine response directed at a broad group of viral proteins. Such a nucleic acid may comprise DNA or RNA or both and may function as an antigenic and immunogenic determinant formulation by allowing expression of the peptides in a cell or vaccinated subject.

In another preferred embodiment, disclosed is a nucleic acid encoding at least 6 of influenza A virus (IAV)-derived peptides capable of inducing IFNγ by CD8+ T cells with amino acid sequences SEQ ID NO:8 (FMYSDFHFI), SEQ ID NO:9 (FVRQCFNPM), SEQ ID NO:12 (NMLSTVLGV), SEQ ID NO:13 (MMMGMFNML), SEQ ID NO:14 (YSHGTGTGY), and SEQ ID NO:19 (RGINDRNFW) and derived from currently circulating human and avian IAV strains to accommodate vaccination against possibly circulating avian influenza A viruses in humans and provide the additional protection desired (see also Tables 2 and 4 herein). In a further preferred embodiment, the disclosure discloses to complement the nucleic acid as provided herein with at least the nucleic acid of 5 of influenza A virus (IAV) derived peptides capable of inducing IFNγ by CD8+ T cells selected from the group of peptides capable of inducing IFNγ by CD8+ T cells with immunodominant amino acid sequences SEQ ID NO:5 (GILGFVFTL), SEQ ID NO:3 (SRYWAIRTR), SEQ ID NO:16 (RRSGAAGAAVK), SEQ ID NO:2 (ELRSRYWAI), and SEQ ID NO:4 (CTELKLSDY) (see also table 5). Such a nucleic acid as provided herein may comprise DNA or RNA or both and may function as an antigenic and immunogenic determinant formulation by allowing expression of the peptides in a cell or vaccinated subject.

In another preferred embodiment, disclosed is a nucleic acid encoding at least 7 of influenza A virus (IAV) derived peptides capable of inducing IFNγ by CD8+ T cells with amino acid sequences SEQ ID NO:8 (FMYSDFHFI), SEQ ID NO:9 (FVRQCFNPM), SEQ ID NO:12 (NMLSTVLGV), SEQ ID NO:13 (MMMGMFNML), SEQ ID NO:14 (YSHGTGTGY), SEQ ID NO:15 (HSNLNDATY), and SEQ ID NO:19 (RGINDRNFW) and derived from currently circulating human, swine and avian IAV strains to accommodate vaccination against possibly circulating swine or avian influenza A viruses in humans and provide the additional protection desired (see also Tables 2, 3 and 4 herein). In a further preferred embodiment, the disclosure discloses to complement the nucleic acid as provided herein with at least the nucleic acid of 5 of influenza A virus (IAV) derived peptides capable of inducing IFNγ by CD8+ T cells selected from the group of peptides capable of inducing IFNγ by CD8+ T cells with immunodominant amino acid sequences SEQ ID NO:5 (GILGFVFTL), SEQ ID NO:3 (SRYWAIRTR), SEQ ID NO:16 (RRSGAAGAAVK), SEQ ID NO:2 (ELRSRYWAI), and SEQ ID NO:4 (CTELKLSDY)(see also table 5). Such a nucleic acid as provided herein may comprise DNA or RNA or both and may function as an antigenic and immunogenic determinant formulation by allowing expression of the peptides in a cell or vaccinated subject.

In a further embodiment, also provided is a nucleic acid encoding at least 10 of influenza A virus (IAV) derived peptides capable of inducing IFNγ by CD8+ T cells selected from the group of peptides capable of inducing IFNγ by CD8+ T cells with amino acid sequences SEQ ID NO:1 (ILRGSVAHK), SEQ ID NO:2 (ELRSRYWAI), SEQ ID NO:3 (SRYWAIRTR), SEQ ID NO:4 (CTELKLSDY), SEQ ID NO:5 (GILGFVFTL), SEQ ID NO:6 (SIIPSGPLK), SEQ ID NO:7 (ASCMGLIY), SEQ ID NO:8 (FMYSDFHFI), SEQ ID NO:9 (FVRQCFNPM), SEQ ID NO:10 (VSDGGPNLY), SEQ ID NO:11 (FLKDVMESM), SEQ ID NO:12 (NMLSTVLGV), SEQ ID NO:13 (MMMGMFNML), SEQ ID NO:14 (YSHGTGTGY), SEQ ID NO:15 (HSNLNDATY), SEQ ID NO:16 (RRSGAAGAAVK), SEQ ID NO:17 (LLTEVETYV), SEQ ID NO:18 (MVLASTTAK), SEQ ID NO:19 (RGINDRNFW) and SEQ ID NO:20 (FLLMDALKL), see, for example, table 8 where selection of a nucleic acid encoding 10 antigenic and immunogenic determinants peptides (SEQ ID NO:1 (ILRGSVAHK), SEQ ID NO:2 (ELRSRYWAI), SEQ ID NO:3 (SRYWAIRTR), SEQ ID NO:4 (CTELKLSDY), SEQ ID NO:5 (GILGFVFTL), SEQ ID NO:6 (SIIPSGPLK), SEQ ID NO:7 (ASCMGLIY), SEQ ID NO:8 (FMYSDFHFI), SEQ ID NO:9 (FVRQCFNPM), and SEQ ID NO:10 (VSDGGPNLY)) provides already HLA coverage of >99%.

In a yet further embodiment, also provided is a nucleic acid encoding at least 15 of influenza A virus (IAV) derived peptides capable of inducing IFNγ by CD8+ T cells selected from the group of peptides capable of inducing IFNγ by CD8+ T cells with amino acid sequences SEQ ID NO:1 (ILRGSVAHK), SEQ ID NO:2 (ELRSRYWAI), SEQ ID NO:3 (SRYWAIRTR), SEQ ID NO:4 (CTELKLSDY), SEQ ID NO:5 (GILGFVFTL), SEQ ID NO:6 (SIIPSGPLK), SEQ ID NO:7 (ASCMGLIY), SEQ ID NO:8 (FMYSDFHFI), SEQ ID NO:9 (FVRQCFNPM), SEQ ID NO:10 (VSDGGPNLY), SEQ ID NO:11 (FLKDVMESM), SEQ ID NO:12 (NMLSTVLGV), SEQ ID NO:13 (MMMGMFNML), SEQ ID NO:14 (YSHGTGTGY), SEQ ID NO:15 (HSNLNDATY), SEQ ID NO:16 (RRSGAAGAAVK), SEQ ID NO:17 (LLTEVETYV), SEQ ID NO:18 (MVLASTTAK), SEQ ID NO:19 (RGINDRNFW) and SEQ ID NO:20 (FLLMDALKL), see, for example, table 8 where selection of a nucleic acid encoding 15 antigenic and immunogenic determinants peptides (SEQ ID NO:1 (ILRGSVAHK), SEQ ID NO:2 (ELRSRYWAI), SEQ ID NO:3 (SRYWAIRTR), SEQ ID NO:4 (CTELKLSDY), SEQ ID NO:5 (GILGFVFTL), SEQ ID NO:6 (SIIPSGPLK), SEQ ID NO:7 (ASCMGLIY), SEQ ID NO:8 (FMYSDFHFI), SEQ ID NO:9 (FVRQCFNPM), SEQ ID NO:10 (VSDGGPNLY), SEQ ID NO:11 (FLKDVMESM), SEQ ID NO:12 (NMLSTVLGV), SEQ ID NO:13 (MMMGMFNML), SEQ ID NO:14 (YSHGTGTGY), and SEQ ID NO:15 (HSNLNDATY)) provides already HLA coverage of >99.5%.

In a further preferred embodiment, also provided is a nucleic acid encoding an antigenic formulation encoding 20 of influenza A virus (IAV) derived peptides capable of inducing IFNγ by CD8+ T cells, in particular, peptides capable of inducing IFNγ by CD8+ T cells with amino acid sequences SEQ ID NO:1 (ILRGSVAHK), SEQ ID NO:2 (ELRSRYWAI), SEQ ID NO:3 (SRYWAIRTR), SEQ ID NO:4 (CTELKLSDY), SEQ ID NO:5 (GILGFVFTL), SEQ ID NO:6 (SIIPSGPLK), SEQ ID NO:7 (ASCMGLIY), SEQ ID NO:8 (FMYSDFHFI), SEQ ID NO:9 (FVRQCFNPM), SEQ ID NO:10 (VSDGGPNLY), SEQ ID NO:11 (FLKDVMESM), SEQ ID NO:12 (NMLSTVLGV), SEQ ID NO:13 (MMMGMFNML), SEQ ID NO:14 (YSHGTGTGY), SEQ ID NO:15 (HSNLNDATY), SEQ ID NO:16 (RRSGAAGAAVK), SEQ ID NO:17 (LLTEVETYV), SEQ ID NO:18 (MVLASTTAK), SEQ ID NO:19 (RGINDRNFW) and SEQ ID NO:20 (FLLMDALKL), see, for example, table 8 and FIG. 6.

In a further preferred embodiment, disclosed is a nucleic acid encoding an antigenic formulation comprising a nucleic acid according to FIG. 6A.

In a further preferred embodiment, disclosed is a nucleic acid encoding an antigenic formulation comprising a nucleic acid according to FIG. 6B.

In a further preferred embodiment, disclosed is a nucleic acid encoding an antigenic formulation comprising a nucleic acid according to FIG. 6C.

In a further preferred embodiment, disclosed is a nucleic acid encoding an antigenic formulation comprising a nucleic acid according to FIG. 6D.

In a further preferred embodiment, the disclosed is a proteinaceous substance at least partly encoded by a nucleic acid encoding an antigenic formulation comprising a nucleic acid according to FIG. 6A.

In a further preferred embodiment, disclosed is a proteinaceous substance at least partly encoded by a nucleic acid encoding an antigenic formulation comprising a nucleic acid according to FIG. 6B.

In a further preferred embodiment, disclosed is a proteinaceous substance at least partly encoded by a nucleic acid or antigenic formulation comprising a nucleic acid according to FIG. 6C.

In a further preferred embodiment, disclosed is a proteinaceous substance at least partly encoded by a nucleic acid encoding an antigenic formulation comprising a nucleic acid according to FIG. 6D.

In a further preferred embodiment, disclosed is an antigenic formulation comprising a proteinaceous substance at least partly encoded by a nucleic acid encoding an antigenic formulation comprising a nucleic acid according to FIG. 6A.

In a further preferred embodiment, disclosed is an antigenic formulation comprising a proteinaceous substance at least partly encoded by a nucleic acid encoding an antigenic formulation comprising a nucleic acid according to FIG. 6B.

In a further preferred embodiment, disclosed is an antigenic formulation comprising a proteinaceous substance at least partly encoded by a nucleic acid encoding an antigenic formulation comprising a nucleic acid according to FIG. 6C.

In a further preferred embodiment, disclosed is an antigenic formulation comprising a proteinaceous substance at least partly encoded by a nucleic acid encoding an antigenic formulation comprising a nucleic acid according to FIG. 6D.

The disclosure also provides a nucleic acid vector (such as pCAGGS as shown herein in the detailed description) comprising nucleic acid according to the disclosure. The disclosure also provides a virus comprising nucleic acid according to the disclosure. The disclosure also provides a cell comprising a nucleic acid or vector or virus according to the disclosure. The disclosure also provides a nucleic acid formulation, such as a DNA or RNA vaccine formulation (see, for example, (Leitner, Ying, and Restifo 1999)), comprising a nucleic acid or vector or virus according to the disclosure. The disclosure also provides a proteinaceous formulation comprising a polyepitope polypeptide derived from a nucleic acid or vector or virus or cell according to the disclosure.

The disclosure also provides a synthetic polyepitope polypeptide or antigenic and immunogenic determinant formulation comprising at least 5, preferably at least 10, more preferably at least 15 of influenza A virus (IAV) derived peptides capable of inducing IFNγ by CD8+ T cells selected from the group of peptides capable of inducing IFNγ by CD8+ T cells with amino acid sequences SEQ ID NO:1 (ILRGSVAHK), SEQ ID NO:2 (ELRSRYWAI), SEQ ID NO:3 (SRYWAIRTR), SEQ ID NO:4 (CTELKLSDY), SEQ ID NO:5 (GILGFVFTL), SEQ ID NO:6 (SIIPSGPLK), SEQ ID NO:7 (ASCMGLIY), SEQ ID NO:8 (FMYSDFHFI), SEQ ID NO:9 (FVRQCFNPM), SEQ ID NO:10 (VSDGGPNLY), SEQ ID NO:11 (FLKDVMESM), SEQ ID NO:12 (NMLSTVLGV), SEQ ID NO:13 (MMMGMFNML), SEQ ID NO:14 (YSHGTGTGY), SEQ ID NO:15 (HSNLNDATY), SEQ ID NO:16 (RRSGAAGAAVK), SEQ ID NO:17 (LLTEVETYV), SEQ ID NO:18 (MVLASTTAK), SEQ ID NO:19 (RGINDRNFW) and SEQ ID NO:20 (FLLMDALKL). Such polyepitope peptides may, for example, comprise conserved human IAV antigenic and immunogenic determinant peptides SEQ ID NO:8 (FMYSDFHFI), SEQ ID NO:9 (FVRQCFNPM), SEQ ID NO:12 (NMLSTVLGV), SEQ ID NO:13 (MMMGMFNML), and SEQ ID NO:14 (YSHGTGTGY), or swine IAV antigenic and immunogenic determinant peptides SEQ ID NO:8 (FMYSDFHFI), SEQ ID NO:12 (NMLSTVLGV), SEQ ID NO:13 (MMMGMFNML), SEQ ID NO:14 (YSHGTGTGY), and SEQ ID NO:15 (HSNLNDATY), or avian IAV antigenic and immunogenic determinant peptides SEQ ID NO:8 (FMYSDFHFI), SEQ ID NO:14 (YSHGTGTGY), SEQ ID NO:13 (MMMGMFNML), SEQ ID NO:12 (NMLSTVLGV), and SEQ ID NO:19 (RGINDRNFW), or SEQ ID NO:8 (FMYSDFHFI), SEQ ID NO:9 (FVRQCFNPM), SEQ ID NO:12 (NMLSTVLGV), SEQ ID NO:13 (MMMGMFNML), SEQ ID NO:14 (YSHGTGTGY), and SEQ ID NO:15 (HSNLNDATY), or SEQ ID NO:8 (FMYSDFHFI), SEQ ID NO:9 (FVRQCFNPM), SEQ ID NO:12 (NMLSTVLGV), SEQ ID NO:13 (MMMGMFNML), SEQ ID NO:14 (YSHGTGTGY), and SEQ ID NO:19 (RGINDRNFW), or SEQ ID NO:8 (FMYSDFHFI), SEQ ID NO:9 (FVRQCFNPM), SEQ ID NO:12 (NMLSTVLGV), SEQ ID NO:13 (MMMGMFNML), SEQ ID NO:14 (YSHGTGTGY), SEQ ID NO:15 (HSNLNDATY), and SEQ ID NO:19 (RGINDRNFW), or immunodominant amino acid sequences SEQ ID NO:5 (GILGFVFTL), SEQ ID NO:3 (SRYWAIRTR), SEQ ID NO:16 (RRSGAAGAAVK), SEQ ID NO:2 (ELRSRYWAI), and SEQ ID NO:4 (CTELKLSDY), or another selection of at least 5 peptides as selected from in Table 1. Peptide synthesis is well known in the art (see, for example, (Stawikowski and Fields 2012)). The disclosure also provides synthetic polyepitope polypeptide or antigenic and immunogenic determinant formulation comprising at least 10 of influenza A virus (IAV) derived peptides capable of inducing IFNγ by CD8+ T cells selected from the group of peptides capable of inducing IFNγ by CD8+ T cells with amino acid sequences SEQ ID NO:1 (ILRGSVAHK), SEQ ID NO:2 (ELRSRYWAI), SEQ ID NO:3 (SRYWAIRTR), SEQ ID NO:4 (CTELKLSDY), SEQ ID NO:5 (GILGFVFTL), SEQ ID NO:6 (SIIPSGPLK), SEQ ID NO:7 (ASCMGLIY), SEQ ID NO:8 (FMYSDFHFI), SEQ ID NO:9 (FVRQCFNPM), SEQ ID NO:10 (VSDGGPNLY), SEQ ID NO:11 (FLKDVMESM), SEQ ID NO:12 (NMLSTVLGV), SEQ ID NO:13 (MMMGMFNML), SEQ ID NO:14 (YSHGTGTGY), SEQ ID NO:15 (HSNLNDATY), SEQ ID NO:16 (RRSGAAGAAVK), SEQ ID NO:17 (LLTEVETYV), SEQ ID NO:18 (MVLASTTAK), SEQ ID NO:19 (RGINDRNFW) and SEQ ID NO:20 (FLLMDALKL).

The disclosure also provides a synthetic polyepitope polypeptide or antigenic and immunogenic determinant formulation comprising at least 15 of influenza A virus (IAV) derived peptides capable of inducing IFNγ by CD8+ T cells selected from the group of peptides capable of inducing IFNγ by CD8+ T cells with amino acid sequences SEQ ID NO:1 (ILRGSVAHK), SEQ ID NO:2 (ELRSRYWAI), SEQ ID NO:3 (SRYWAIRTR), SEQ ID NO:4 (CTELKLSDY), SEQ ID NO:5 (GILGFVFTL), SEQ ID NO:6 (SIIPSGPLK), SEQ ID NO:7 (ASCMGLIY), SEQ ID NO:8 (FMYSDFHFI), SEQ ID NO:9 (FVRQCFNPM), SEQ ID NO:10 (VSDGGPNLY), SEQ ID NO:11 (FLKDVMESM), SEQ ID NO:12 (NMLSTVLGV), SEQ ID NO:13 (MMMGMFNML), SEQ ID NO:14 (YSHGTGTGY), SEQ ID NO:15 (HSNLNDATY), SEQ ID NO:16 (RRSGAAGAAVK), SEQ ID NO:17 (LLTEVETYV), SEQ ID NO:18 (MVLASTTAK), SEQ ID NO:19 (RGINDRNFW) and SEQ ID NO:20 (FLLMDALKL).

The disclosure also provides an antigenic and immunogenic determinant or immunogenic formulation (see, for example, FIGS. 2 and 3 where various constructs (Polyepitope, polyepitope with spacers, polyepitope with ubiquitin and polyepitope with spacer and ubiquitin) activated the specific T cells and IFN-γ secretion) comprising a nucleic acid according to the disclosure or comprising a virus according to the disclosure or comprising a cell according to the disclosure or comprising a proteinaceous formulation according to the disclosure or a synthetic polyepitope polypeptide or antigenic and immunogenic determinant formulation according to the disclosure. The disclosure also provides an immunogenic formulation comprising a nucleic acid formulation according to the disclosure or a proteinaceous formulation according to the disclosure or a synthetic polyepitope polypeptide or antigenic and immunogenic determinant formulation according to the disclosure and provides a vaccine formulation obtainable by mixing an antigenic and immunogenic determinant formulation according to the disclosure and/or an immunogenic formulation according to the disclosure and a pharmaceutically acceptable excipient.

The disclosure also provides a vaccine formulation according to the disclosure for use in vaccination together with or in addition to or preferably concomitant with, preferably yearly, vaccination with a vaccine directed at generating humoral vaccine responses toward an influenza virus hemagglutinin protein. In a preferred embodiment, to improve on and provide an alternative influenza vaccine with efficacy in circumstances of relative mismatch between circulating influenza strains and vaccine strains used, vaccinating with an influenza vaccine in addition to or preferably concomitant with yearly vaccination with trivalent or quadrivalent vaccines directed at humoral vaccine responses toward the hemagglutinin protein is proposed. It is preferred that the influenza vaccine elicits immune responses toward conserved influenza virus antigens, in particular, by providing the vaccine with immunogenic formulations comprising or capable of in vivo eliciting immune responses directed against peptides capable of inducing IFNγ by CD8+ T cells selected from the group of peptides capable of inducing IFNγ by CD8+ T cells with amino acid sequences SEQ ID NO:1 (ILRGSVAHK), SEQ ID NO:2 (ELRSRYWAI), SEQ ID NO:3 (SRYWAIRTR), SEQ ID NO:4 (CTELKLSDY), SEQ ID NO:5 (GILGFVFTL), SEQ ID NO:6 (SIIPSGPLK), SEQ ID NO:7 (ASCMGLIY), SEQ ID NO:8 (FMYSDFHFI), SEQ ID NO:9 (FVRQCFNPM), SEQ ID NO:10 (VSDGGPNLY), SEQ ID NO:11 (FLKDVMESM), SEQ ID NO:12 (NMLSTVLGV), SEQ ID NO:13 (MMMGMFNML), SEQ ID NO:14 (YSHGTGTGY), SEQ ID NO:15 (HSNLNDATY), SEQ ID NO:16 (RRSGAAGAAVK), SEQ ID NO:17 (LLTEVETYV), SEQ ID NO:18 (MVLASTTAK), SEQ ID NO:19 (RGINDRNFW) and SEQ ID NO:20 (FLLMDALKL). Preferably the responses are cellular immune responses, therewith supplementing humoral responses, e.g., directed at the variable hemagglutinin. Most preferred cellular immune responses elicited are CD8+-CTL responses as provided herein.

### BRIEF DESCRIPTION OF THE DRAWINGS

FIG. 1: Schematic diagram of enrichment of peptide specific CD8+ T cells from blood. Isolated PBMCs from blood are incubated with 10 μM specific peptide, after 3 days added IL-2. After 9 days of adding IL-2, CD8+ T-cells were isolated by using MACS.

FIG. 2: Antigenicity test in vitro with pCAGGS constructs as antigenic formulation. PB1₃₀₋₃₉ (SEQ ID NO:14 (YSHGTGTGY))-specific CD8+ T cells incubated with transfected HLA*B15 transgenic A549 cells. All constructs (polyepitope, polyepitope with spacers, polyepitope with ubiquitin and polyepitope with spacer and ubiquitin) activated the specific T cells and IFN-γ secretion. PB1₃₀₋₃₈ peptide loaded cells are used as a positive control. Non-transfected T cells are used a negative control as well as empty vector transfected and untransfected transgenic A549-B*15.

FIG. 3: Antigenicity test in vitro with recombinant MVA constructs as antigenic formulation. PB1₃₀₋₃₈ (SEQ ID NO:14 (YSHGTGTGY))-specific CD8+ T cells incubated with infected HLA*B15 transgenic A549 cells with two different MOIs (1 and 3). Only one polyepitope construct with ubiquitin and with spacer activated the specific T cells and IFN-γ secretion. PB1₃₀₋₃₈ peptide loaded cells are used as a positive control. Only T cells are used a negative control as well as non-recombinant MVA and uninfected transgenic A549-B*15 cells.

FIG. 4: Schematic representation of various construct designs used. Four polyepitope constructs were made with or without spacer and with or without ubiquitin. One construct was made by complete M1 gene. All constructs were fused with HA. The addition of an HA epitope serves for easy monitoring of gene expression. All five constructs were cloned in pCAGGS. Construction of artificial polyepitope genes as antigenic formulation. The effect of the addition of ubiquitin and spacer sequence on antigen processing and presentation of the individual epitopes is investigated.

FIG. 6A: Polyepitope constructs useful as antigenic and immunogenic determinant formulation: artificial gene cloned into:
pCAGGS
MVA shuttle vector (pH5 promotor)
Optimized Polyepitope (SEQ ID NO:46) without spacer (599 bp)

FIG. 6B: Polyepitope constructs useful as antigenic and immunogenic determinant formulation: artificial gene cloned into:
pCAGGS
MVA shuttle vector (pH5 promotor)
Optimized Polyepitope (SEQ ID NO:45) with spacers: (770 bp)

FIG. 6C: Polyepitope constructs useful as antigenic and immunogenic determinant formulation: artificial gene cloned into:
pCAGGS
MVA shuttle vector (pH5 promotor)
Optimized Polyepitope (SEQ ID NO:47) without spacers with ubiquitin (823 bp)

FIG. 6D: Polyepitope constructs useful as antigenic and immunogenic determinant formulation: artificial gene cloned into:
pCAGGS
MVA shuttle vector (pH5 promotor)
Optimized Polyepitope (SEQ ID NO:48) with spacers and ubiquitin (995 bp)

FIG. 7

Polyepitope constructs cloned into pCAGGS and MVA useful as antigenic and immunogenic determinant formulation.

With the pCAGGS expression plasmids, an IFN-γ response in the enriched T cell populations for all four epitopes tested was observed, indicating that the epitopes could be liberated from the polyepitope construct and presented to specific T cells. With the MVA constructs, an IFN-γ response in the enriched T cell populations only for the construct with ubiquitin and spacer was observed, indicating that the epitopes could be liberated from the polyepitope construct and presented to specific T cells. In MVA, only 2 constructs were construed and tested.

FIG. 8

Immunogenicity in vitro of rMVA-PE, wt MVA and IAV. PBMCs of a HLA-A*01 positive blood donor were stimulated with rMVA-PE, wt MVA or IAV (moi of 3) and after 12 days of T cell expansion, the presence of IAV epitope specific T cells was assessed by IFN-γ ELISpot assay after restimulation with HLA-A*01 transgenic A549 cell loaded with peptides SEQ ID NO:4 (CTELKLSDY) (NP₄₄₋₅₂, CTE) and SEQ ID NO:10 (VSDGGPNLY)(PB1₅₉₁₋₅₉₉, VSD) or control cells.

FIG. 9

Experimental vaccination scheme

FIG. 10

Description of vaccinated groups.

FIG. 11

Immunogenicity test in vivo with recombinant MVA constructs. Spleens were harvested two weeks after the booster vaccination and splenocytes were restimulated with HLA-A*02-restricted IAV M1₅₈₋₆₆ peptide with SEQ ID NO:5 (GILGFVFTL) (FIG. 11 at left) or with MVA-derived A6L peptide (FIG. 11 at right). The data are expressed as the numbers of IFN-γ-spot forming units per 10⁶ splenocytes after background subtraction as determined by ELISpot assay.

### DETAILED DESCRIPTION

Influenza viruses cause yearly epidemics of mild disease and, occasionally, pandemics with millions of fatalities. Currently, no vaccine is effective against all influenza strains. Extraordinary genetic variability and continuous evolution are responsible for the virus evading immunity in the host and necessitating annual update of the seasonal vaccine. Since the majority of virus-specific T cells, and, in particular, CD8+ cytotoxic T lymphocytes (CTL), are directed against relatively conserved viral proteins like the nucleoprotein (NP) and the matrix 1 protein (M1), it has been suggested already many decades ago that virus-specific CTLs may contribute to heterosubtypic immunity (Effros et al. 1977). The most important mode of action of virus-specific CTL is recognition and elimination of virus-infected cells. This way, the production of progeny virus is prevented. Thus, the presence of pre-existing T-cell immunity results in more rapid clearance of virus infections. Key for heterosubtypic immunity is that CTLs are cross-reactive and recognize epitopes shared by influenza A viruses of different subtypes. The effectors functions of CTLs that are responsible for the elimination of virus-infected cells include the release of perforin and granzyme from their granules and Fas/FasL interactions with infected target cells. In addition, upon activation virus-specific CD8⁺ T cells can produce a variety of different cytokines including IFN-γ and TNF-α. It is shown that virus-specific CTLs through their receptor recognize viral peptides, which are generated by the endogenous route of antigen processing and that are ultimately presented by MHC class I molecules on the surface of antigen-presenting cells or virus-infected cells (Zammit et al. 2005). With this specific focus on inducing CD8+-CTL-responses, the disclosure avoids reduced efficacies, which are, for example, observed with polypeptide epitope vaccine formulations M-001 and FLU-v.

### Generation of Recombinant Viruses.

The coding sequence of each of 20 selected epitopes (peptide sequences are given in the one-letter amino acid code, see table 1) and the spacers in between the epitopes are composed in silico. For expedient transcription and/or translation, these are preferable modified by introducing silent mutations to remove runs of guanines or cytosines and termination signals of vaccinia virus-specific early transcription, and to add a C-terminal HA-tag sequence encoding nine amino acids (SEQ ID NO:21 (YPYDVPDYA), amino acids 98-106 from influenza virus). Ubiquitin may also be added to N-terminal in a polyepitope construct. The cDNA is produced by DNA synthesis and cloned into a vector such as the MVA transfer plasmid pIIIH5red (Song et al. 2013) under transcriptional control of the synthetic vaccinia virus early/late promoter PmH5. MVA (clonal isolate MVA-F6) is grown on CEFs under serum-free conditions and may serve as a nonrecombinant backbone virus to construct MVA vector viruses expressing the desired polyepitope gene sequences. To obtain vaccine preparations, recombinant polyepitopes may, for example, be amplified on CEF monolayers, purified by ultracentrifugation on a sucrose bed and reconstituted to high-titer stock preparations. PFUs may be counted to determine viral titers.

### Study Subjects

Blood obtained from healthy blood donors are used in a first study. Blood collected between April and August 2019 at the Blood bank of the Hannover Medical School (Medizinische Hochschule Hannover, MHH) is used. The use of blood for scientific purposes is approved by the local MHH ethical committee and the subjects gave written informed consent and data have been used in an anonymized form. The work described here has been carried out in accordance with the code of ethics of the world medical association (declaration of Helsinki).

### Cell Culture.

Chicken embryo fibroblasts (CEFs or CEF cells) are isolated from 10-day-old chicken embryos (Valo BioMedia GmbH, Germany) and passaged once before use. CEFs are cultured in Minimum Essential Medium Eagle with Earle's salts, L-glutamine, and sodium bicarbonate (MEME; Sigma) supplemented with 10% FBS, 1% penicillin and streptomycin (P/S) and 1% non-essential amino acid (NEAA). A549 cells are cultured in F-12K nutrient mix medium (Gibco) supplemented with 10% FBS, 1% P/S, 1% Glutamax and transgenic A549 cells are cultured in F-12K nutrient mix medium (Gibco) supplemented with 10% FBS, 1% P/S, 1% Glutamax and 1 μg/ml puromycin. All cell lines are cultured at 37° C. with 5% CO2.

### Isolation of PBMCs

Peripheral blood mononuclear cells (PBMCs) are isolated from peripheral blood by density gradient centrifugation (Lymphoprep; Stem cell) according to the manufacturer's instructions. Cells are isolated, then counted and frozen in 90% fetal bovine serum (FBS, Thermo Fisher), 10% dimethyl sulfoxide (DMSO, Carl Roth), and cryopreserved in liquid nitrogen or at −150° C. until use. The PBMCs are thawed in complete RPMI 1640 medium, supplemented with penicillin/streptomycin, Glutamax, vitamins, non-essential aminoacids, sodium pyruvate (all at 1% v/v, Thermo Fisher), 10% (v/v) heat-inactivated Fetal Bovine Serum (FBS; Gibco Thermo Fisher) (R10F) and 50 μg/ml of DNAse (Sigma Aldrich).

### Enrichment of Peptide Specific CD8+ T Cells.

For the enrichment of peptide specific CD8+ T-cells, peptide specific T cells are expanded using PBMCs from healthy HLA matched blood donor incubated with 10 μM corresponding peptide at 37° C., 5% CO₂. After 3 days, IL-2 is added. After 9 days of expansion, the peptide specific CD8+ T cells are detected by fluorochrome-conjugated CD8+ staining and flowcytometry. By using CD8+ specific beads, CD8+ stained cells were isolated by using Magnetic-activated cell sorting (MACS).

### Detection of Virus-Specific T Cells by IFN-γ ELISpot Assay

Human IFN-γ ELISpot kits (Mabtech) containing 96 well pre-coated plates and the assay are carried out according to the manufacturer's instructions. Isolated CD8+ T cells are co-cultured with infected, or peptide loaded HLA transgenic A549 for 20 h at 37° C., 5% CO2 and IFN-γ secretion in response to peptide specific T-cell activation is measured by ELISpot. Only transgenic A549 cells incubated with peptide or only CD8+ T cells are used as positive and negative controls, respectively. After development, plates are scanned, and spots counted using the ImmunoSpot S6 Ultimate Reader and ImmunoSpot Software (Version 7.0.20.0, Immunospot, CTL).

The Immunoepitope database (IEDB) (Bui et al. 2007), contains 972 unique influenza virus CD8+ T cell epitopes (as accessed September 2020). From these, 20 epitopes are selected based on the conservation of the respective epitopes in influenza viruses from various hosts and antigenicity of the epitopes confirmed.

### Abilities to Direct IFN-Gamma Secretion by T-Cells

As a minimum inclusion criterion, the antigenicity of the epitopes (antigenic and immunogenic determinants) are confirmed by determining that peptides corresponding to the epitopes expressed by the antigenic formulation can induce interferon-gamma (IFNγ) secretion by virus specific CD8+ T cells, for example, as demonstrated by IFNγ ELIspot assay or intracellular cytokine staining.

Conservation of the Epitope within Human, Avian and Swine Influenza A Strains

While aiming for universal vaccine, it is very important to select those epitopes, which are very conserved not only in human but also in other species. Humans can be infected with avian, swine and other zoonotic influenza viruses, such as avian influenza virus subtypes A(H5N1), A(H7N9), and A(H9N2) and swine influenza virus subtypes A(H1N1), A(H1N2) and A(H3N2). With the peptides in the current 20-epitope polyepitope construct, a sequence analysis was completed and showed the peptides how conserved they are in human, avian and swine flu viruses.

Sequence Collected from NCBI Flu Database
Breadth of Fit within the Human HLA System

When a cell, encounters an antigen, if T cells recognize it as a ‘normal antigen’, nothing will happen. However, if they recognize as a foreign or pathologic antigen, T cells will be activated. To be recognized by T cells, antigens must be loaded on human leucocyte antigen (HLA) molecules. In human, the major histocompatibility complex (MHC) of vertebrates, a group of cell-surface proteins coded by more than 250 genes on chromosome 6, are termed as HLA. All of us inherit several HLA genes from our parents. HLA types are not evenly distributed among the population, as some HLA types are more frequent than others are. For example: The HLA-A2 family is the largest allele family of the HLA-A locus (Bodmer et al. 1999)

TABLE 1
Epitopes, listed from N-terminal to C-terminal of expressed polyepitope
construct with HLA coverage and conservation of the epitope in human, avian and swine:

| | | | Conserved in influenza virus | | |
|---|---|---|---|---|---|
| CD8+ Epitope | Sequence | HLA | Human | Avian | Swine |
| NP ₂₆₅₋₂₇₄ | SEQ ID NO: 1 | HLA-A*03:01 | 98% | 94% | 97.7% |
| | (ILRGSVAHK) | | | | |
| NP ₃₈₀₋₃₈₈ | SEQ ID NO: 2 | HLA-A*02:01, HLA-A*69:01, HLA- | 56% | 94% | 72% |
| | (ELRSRYWAI) | B*18:01, HLA-B*27:02 | | | |
| NP ₃₈₃₋₃₉₁ | SEQ ID NO: 3 | HLA-B*27:05, HLA-B*27, HLA- | 55.9% | 92% | 72% |
| | (SRYWAIRTR) | B*27:03, HLA-B*27:04, HLA- | | | |
| | | B*27:06, HLA-B*27:02, HLA- | | | |
| | | B*27:09 | | | |
| NP ₄₄₋₅₂ | SEQ ID NO: 4 | HLA-A*01:01, HLA-A*30:03, SLA- | 55.5% | 64.7% | 72% |
| | (CTELKLSDY) | 1*04:01, HLA-B*15:17 | | | |
| M1 ₅₈₋₆₆ | SEQ ID NO: 5 | HLA-A*02:01, HLA-A*02:06, HLA- | 97% | 85% | 96.9% |
| | (GILGFVFTL) | A*02:02, HLA-A*02:03, HLA- | | | |
| | | C*08:01, HLA-A*68:02, HLA- | | | |
| | | E*01:03 | | | |
| M1 ₁₃₋₂₁ | SEQ ID NO: 6 | HLA-A*11:01, HLA-A*03:01, HLA- | 55% | 54% | 65.6% |
| | (SIIPSGPLK) | A*31:01, HLA-A*68:01 | | | |
| M1 ₁₂₈₋₁₃₅ | SEQ ID NO: 7 | HLA-B*35:01 | 96.6% | 94% | 96.9% |
| | (ASCMGLIY) | | | | |
| PA ₄₆₋₅₁ | SEQ ID NO: 8 | HLA-A*02:01, HLA-A*02:02, HLA- | 99.2% | 98.9% | 99% |
| | (FMYSDFHFI) | A*02:03, HLA-A*02:06, HLA- | | | |
| | | A*68:02, HLA-A*24:02, HLA- | | | |
| | | A*69:01, HLA-C*07:02 | | | |
| PA ₄₋₁₂ | SEQ ID NO: 9 | HLA-B*15:01, HLA-A*02:01, HLA- | 98% | 97.8% | 98% |
| | (FVRQCFNPM) | B*07:02, HLA-B*08:01, HLA- | | | |
| | | C*14:02 | | | |
| PB1 ₅₉₁₋₅₉₉ | SEQ ID NO: 10 | HLA-A*01:01, HLA-A*29:02, HLA- | 97.9% | 95.8% | 92% |
| | (VSDGGPNLY) | A*30:02, HLA-A*80:01, HLA- | | | |
| | | B*15:01, HLA-B*15:17, HLA- | | | |
| | | B*58:01, HLA-C*08:02, | | | |
| PB1 ₁₆₆₋₁₇₄ | SEQ ID NO: 11 | HLA-A*02:01, HLA-B*07:02, HLA- | 93.9% | 78.8% | 85% |
| | (FLKDVMESM) | B*15:01, HLA-B*08:01, HLA- | | | |
| | | A*02:11, HLA-A*26:01, HLA-A*02, | | | |
| | | HLA-B*46:01, HLA-C*12:03 | | | |
| PB1 ₄₁₃₋₄₂₁ | SEQ ID NO: 12 | HLA-A*02:01, HLA-A*02:02, HLA- | 98.9% | 98.6% | 98.6% |
| | (NMLSTVLGV) | A*02:03, HLA-A*02:06, HLA- | | | |
| | | A*68:02 | | | |
| PB1 ₄₀₇₋₄₁₅ | SEQ ID NO: 13 | HLA-A*02:01, HLA-A*02:02, HLA- | 99% | 98.6% | 98.8% |
| | (MMMGMFNML) | A*02:03, HLA-A*02:06, HLA- | | | |
| | | A*68:02, HLA-A*24:02, HLA- | | | |
| | | A*69:01, HLA-B*15:01 | | | |
| PB1 ₃₀₋₃₈ | SEQ ID NO: 14 | HLA-A*26:01, HLA-B*15:01, HLA- | 99% | 99.5% | 99% |
| | (YSHGTGTGY) | A*01:01, HLA-A*29:02, HLA- | | | |
| | | A*30:02, HLA-B*58:01 | | | |
| NP ₁₄₀₋₁₄₈ | SEQ ID NO: 15 | HLA-A*01:01, HLA-A*26:01, HLA- | 83% | 96.7% | 96.6% |
| | (HSNLNDATY) | B*15:01, HLA-A*30:02, HLA- | | | |
| | | A*80:01, HLA-B*15:17, HLA- | | | |
| | | B*35:01, HLA-B*57:01, HLA- | | | |
| | | B*58:01 | | | |
| NP ₁₇₄₋₁₈₄ | SEQ ID NO: 16 | HLA-B*27:01, HLA-B*27:05 | 96.6% | 93.5% | 96.5% |
| | (RRSGAAGAAVK) | | | | |
| M1 ₃₋₁₁ | SEQ ID NO: 17 | HLA-A*02:01, HLA-A*02:03, HLA- | 96% | 93.9% | 97.7% |
| | (LLTEVETYV) | A*02:06, HLA-A*02:11, HLA- | | | |
| | | A*02:12, HLA-A*02:16, HLA- | | | |
| | | A*02:19 | | | |
| M1 ₁₇₉₋₁₈₇ | SEQ ID NO: 18 | HLA-A*11:01, HLA-A*03:01, HLA- | 97% | 94.7% | 79% |
| | (MVLASTTAK) | A*31:01 | | | |
| NP ₁₉₉₋₂₀₇ | SEQ ID NO: 19 | HLA-B*58:01 | 98.6% | 91.9% | 98.9% |
| | (RGINDRNFW) | | | | |
| PA ₂₈₂₋₂₉₀ | SEQ ID NO: 20 | HLA-A*02:01, HLA-A*02:02, HLA- | 93.9% | 96.7% | 98% |
| | (FLLMDALKL) | A*02:03, HLA-A*02:06, HLA- | | | |
| | | A*02:11, HLA-A*02:12, HLA- | | | |
| | | A*69:01 | | | |

*Represent the proportion of viruses in the influenza virus sequence data base (ncbi.nlm.nih.gov/genomes/FLU/Database/nph-select.cgi?go=database) with the indicated epitope sequence without any mutations. For this analysis duplicate sequences are excluded.

### HLA Coverage on World Poppulation:

T cells can recognize peptides only if they are bound to HLA molecules. These peptide-HLA complexes interact only with HLA-matched T cells that have the corresponding receptors. To prepare a peptide-based vaccine as a universal one, the main goal is to have a very high HLA coverage. Here in the table below you can see the number of epitopes and their combined HLA coverage range in percentage. HLA coverage calculation is done in IEDB database.

TABLE 2
Set 1
According to highest conserved epitope in human from the 20 epitope list
3 out of below 5 epitopes are not only conserved in IAV but also in IBV
Giving a good world population HLA coverage

| Antigen | | | | | | |
|---|---|---|---|---|---|---|
| (amino acid | | Restriction | Conserved in influenza virus | | | HLA |
| positions) | Epitope sequence | element | Human | Avian | Swine | coverage |
| PA 46-51 | SEQ ID NO: 8 | HLA-A*02:01, | 99.2% | 98.9% | 99% | 96.81% |
| | (FMYSDFHFI) | HLA-A*02:02, | | | | |
| | | HLA-A*02:03, | | | | |
| | | HLA-A*02:06, | | | | |
| | | HLA-A*68:02, | | | | |
| | | HLA-A*24:02, | | | | |
| | | HLA-A*69:01, | | | | |
| | | HLA-C*07:02 | | | | |
| PA 4-12 | SEQ ID NO: 9 | HLA-B*15:01, | 98% | 97.8% | 98% | |
| | (FVRQCFNPM) | HLA-A*02:01, | | | | |
| | | HLA-B*07:02, | | | | |
| | | HLA-B*08:01, | | | | |
| | | HLA-C*14:02 | | | | |
| PB1 413-421 | SEQ ID NO: 12 | HLA-A*02:01, | 98.9% | 98.6% | 98.6% | |
| | (NMLSTVLGV) | HLA-A*02:02, | | | | |
| | | HLA-A*02:03, | | | | |
| | | HLA-A*02:06, | | | | |
| | | HLA-A*68:02 | | | | |
| PB1 407-415 | SEQ ID NO: 13 | HLA-A*02:01, | 99% | 98.6% | 98.6% | |
| | (MMMGMFNML) | HLA-A*02:02, | | | | |
| | | HLA-A*02:03, | | | | |
| | | HLA-A*02:06, | | | | |
| | | HLA-A*68:02, | | | | |
| | | HLA-A*24:02, | | | | |
| | | HLA-A*69:01, | | | | |
| | | HLA-B*15:01 | | | | |
| PB1 30-38 | SEQ ID NO: 14 | HLA-A*26:01, | 99% | 99.5% | 99% | |
| | (YSHGTGTGY) | HLA-B*15:01, | | | | |
| | | HLA-A*01:01, | | | | |
| | | HLA-A*29:02, | | | | |
| | | HLA-A*30:02, | | | | |
| | | HLA-B*58:01 | | | | |

TABLE 3
Set 2
According to highest conserved epitope in avian from the 20 epitope list
3 out of below 5 epitopes are not only conserved in IAV but also in IBV
Giving a good world population HLA coverage

| Antigen | | | | | | |
|---|---|---|---|---|---|---|
| (amino acid | | Restriction | Conserved in influenza virus | | | HLA |
| positions) | Epitope sequence | element | Human | Avian | Swine | coverage |
| PA 46-51 | SEQ ID NO: 8 | HLA-A*02:01, | 99.2% | 98.9% | 99% | 85.38% |
| | (FMYSDFHFI) | HLA-A*02:02, | | | | |
| | | HLA-A*02:03, | | | | |
| | | HLA-A*02:06, | | | | |
| | | HLA-A*68:02, | | | | |
| | | HLA-A*24:02, | | | | |
| | | HLA-A*69:01, | | | | |
| | | HLA-C*07:02 | | | | |
| PB1 413-421 | SEQ ID NO: 12 | HLA-A*02:01, | 98.9% | 98.6% | 98.6% | |
| | (NMLSTVLGV) | HLA-A*02:02, | | | | |
| | | HLA-A*02:03, | | | | |
| | | HLA-A*02:06, | | | | |
| | | HLA-A*68:02 | | | | |
| PB1 407-415 | SEQ ID NO: 13 | HLA-A*02:01, | 99% | 98.6% | 98.6% | |
| | (MMMGMFNML) | HLA-A*02:02, | | | | |
| | | HLA-A*02:03, | | | | |
| | | HLA-A*02:06, | | | | |
| | | HLA-A*68:02, | | | | |
| | | HLA-A*24:02, | | | | |
| | | HLA-A*69:01, | | | | |
| | | HLA-B*15:01 | | | | |
| PB1 30-38 | SEQ ID NO: 14 | HLA-A*26:01, | 99% | 99.5% | 99% | |
| | (YSHGTGTGY) | HLA-B*15:01, | | | | |
| | | HLA-A*01:01, | | | | |
| | | HLA-A*29:02, | | | | |
| | | HLA-A*30:02, | | | | |
| | | HLA-B*58:01 | | | | |
| NP 140-148 | SEQ ID NO: 15 | HLA-A*01:01, | 83% | 96.7% | 96.6% | |
| | (HSNLNDATY) | HLA-A*26:01, | | | | |
| | | HLA-B*15:01, | | | | |
| | | HLA-A*30:02, | | | | |
| | | HLA-A*80:01, | | | | |
| | | HLA-B*15:17, | | | | |
| | | HLA-B*35:01, | | | | |
| | | HLA-B*57:01, | | | | |
| | | HLA-B*58:01 | | | | |

TABLE 4
Set 3
According to highest conserved epitope in swine from the 20-epitope list
2 out of below 5 epitopes are not only conserved in IAV but also in IBV
Giving a good world population HLA coverage

| Antigen | | | | | | |
|---|---|---|---|---|---|---|
| (amino acid | | Restriction | Conserved in influenza virus | | | HLA |
| positions) | Epitope sequence | element | Human | Avian | Swine | coverage |
| PA 46-51 | SEQ ID NO: 8 | HLA- | 99.2% | 98.9% | 99% | 84.86% |
| | (FMYSDFHFI) | A*02:01, | | | | |
| | | HLA- | | | | |
| | | A*02:02, | | | | |
| | | HLA- | | | | |
| | | A*02:03, | | | | |
| | | HLA- | | | | |
| | | A*02:06, | | | | |
| | | HLA- | | | | |
| | | A*68:02, | | | | |
| | | HLA- | | | | |
| | | A*24:02, | | | | |
| | | HLA- | | | | |
| | | A*69:01, | | | | |
| | | HLA-C*07:02 | | | | |
| PB1 30-38 | SEQ ID NO: 14 | HLA- | 99% | 99.5% | 99% | |
| | (YSHGTGTGY) | A*26:01, | | | | |
| | | HLA-B*15:01, | | | | |
| | | HLA- | | | | |
| | | A*01:01, | | | | |
| | | HLA- | | | | |
| | | A*29:02, | | | | |
| | | HLA- | | | | |
| | | A*30:02, | | | | |
| | | HLA-B*58:01 | | | | |
| PB1 407-415 | SEQ ID NO: 13 | HLA- | 99% | 98.6% | 98.6% | |
| | (MMMGMFNML) | A*02:01, | | | | |
| | | HLA- | | | | |
| | | A*02:02, | | | | |
| | | HLA- | | | | |
| | | A*02:03, | | | | |
| | | HLA- | | | | |
| | | A*02:06, | | | | |
| | | HLA- | | | | |
| | | A*68:02, | | | | |
| | | HLA- | | | | |
| | | A*24:02, | | | | |
| | | HLA- | | | | |
| | | A*69:01, | | | | |
| | | HLA-B*15:01 | | | | |
| PB1 413-421 | SEQ ID NO: 12 | HLA- | 98.9% | 98.6% | 98.6% | |
| | (NMLSTVLGV) | A*02:01, | | | | |
| | | HLA- | | | | |
| | | A*02:02, | | | | |
| | | HLA- | | | | |
| | | A*02:03, | | | | |
| | | HLA- | | | | |
| | | A*02:06, | | | | |
| | | HLA-A*68:02 | | | | |
| NP 199-207 | SEQ ID NO: 19 | HLA-B*58:01 | 98.6% | 91.9% | 98.9% | |
| | (RGINDRNFW) | | | | | |

TABLE 5
Set 4
According to hierarchy of immunodominance of influenza A virus-specific
CTL (according to (Boon et al. 2006))

| Antigen | | | | | | |
|---|---|---|---|---|---|---|
| (amino acid | | Restriction | Conserved in influenza virus | | | HLA |
| positions) | Epitope sequence | element | Human | Avian | Swine | coverage |
| M1 58-66 | SEQ ID NO: 5 | HLA-A*02:01, | 97% | 85% | 96.9% | 67.46% |
| (Highly | (GILGFVFTL) | HLA-A*02:06, | | | | |
| immunodom | | HLA-A*02:02, | | | | |
| iant) | | HLA-A*02:03, | | | | |
| | | HLA-C*08:01, | | | | |
| | | HLA-A*68:02, | | | | |
| | | HLA-E*01:03 | | | | |
| NP 383-391 | SEQ ID NO: 3 | HLA-B*27:05, | 55.9% | 92% | 72% | |
| (Highly | (SRYWAIRTR) | HLA-B*27:03, | | | | |
| immunodomiant) | | HLA-B*27:04, | | | | |
| | | HLA-B*27:06, | | | | |
| | | HLA-B*27:02, | | | | |
| | | HLA-B*27:09 | | | | |
| NP 174-184 | SEQ ID NO: 16 | HLA-B*27:01, | 96.6% | 93.5% | 96.5% | |
| (Highly | (RRSGAAGAAV | HLA-B*27:05 | | | | |
| immunodominant) | K) | | | | | |
| NP 380-388 | SEQ ID NO: 2 | HLA-A*02:01, | 56% | 94% | 72% | |
| (Intermediate | (ELRSRYWAI) | HLA-A*69:01, | | | | |
| immunodominant) | | HLA-B*18:01, | | | | |
| | | HLA-B*27:02, | | | | |
| | | HLA-B*08:01 | | | | |
| NP 44-52 | SEQ ID NO: 4 | HLA-A*01:01, | 55.5% | 64.7% | 72% | |
| (Intermediate | (CTELKLSDY) | HLA-A*30:03, | | | | |
| immunodominant) | | SLA-1*04:01, | | | | |
| | | HLA-B*15:17 | | | | |

TABLE 6
SET 5
5 epitopes, which gives a good HLA coverage of the world population

| Antigen | | | | | | |
|---|---|---|---|---|---|---|
| (amino acid | | Restriction | Conserved in influenza virus | | | HLA |
| positions) | Epitope sequence | element | Human | Avian | Swine | coverage |
| M1 58-66 | SEQ ID NO: 5 | HLA-A*02:01, | 97% | 85% | 96.9% | 95.15% |
| | (GILGFVFTL) | HLA-A*02:06, | | | | |
| | | HLA-A*02:02, | | | | |
| | | HLA-A*02:03, | | | | |
| | | HLA-C*08:01, | | | | |
| | | HLA-A*68:02, | | | | |
| | | HLA-E*01:03 | | | | |
| PB1 591-599 | SEQ ID NO: 10 | HLA-A*01:01, | 97.9% | 95.8% | 92% | |
| | (VSDGGPNLY) | HLA-A*29:02, | | | | |
| | | HLA-A*30:02, | | | | |
| | | HLA-A*80:01, | | | | |
| | | HLA-B*15:01, | | | | |
| | | HLA-B*15:17, | | | | |
| | | HLA-B*58:01, | | | | |
| | | HLA-C*08:02 | | | | |
| NP 265-274 | SEQ ID NO: 1 | HLA-A*03:01 | 98% | 94% | 97.7% | |
| | (ILRGSVAHK) | | | | | |
| NP 380-388 | SEQ ID NO: 2 | HLA-B*08:01, | 56% | 94% | 72% | |
| | (ELRSRYWAI) | HLA-A*02:01, | | | | |
| | | HLA-A*69:01, | | | | |
| | | HLA-B*18:01, | | | | |
| | | HLA-B*27:02 | | | | |
| NP 383-391 | SEQ ID NO: 3 | HLA-B*27:05, | 55.9% | 92% | 72% | |
| | (SRYWAIRTR) | HLA-B*27:03, | | | | |
| | | HLA-B*27:04, | | | | |
| | | HLA-B*27:06, | | | | |
| | | HLA-B*27:02, | | | | |
| | | HLA-B*27:09 | | | | |

TABLE 7
Set 6:
5 epitopes from different antigens of IAV

| Antigen | | | | | | |
|---|---|---|---|---|---|---|
| (amino acid | | Restriction | Conserved in influenza virus | | | HLA |
| positions) | Epitope sequence | element | Human | Avian | Swine | coverage |
| M1 58-66 | SEQ ID NO: 5 | HLA-A*02:01, | 97% | 85% | 96.9% | 93.48% |
| | (GILGFVFTL) | HLA-A*02:06, | | | | |
| | | HLA-A*02:02, | | | | |
| | | HLA-A*02:03, | | | | |
| | | HLA-C*08:01, | | | | |
| | | HLA-A*68:02, | | | | |
| | | HLA-E*01:03 | | | | |
| PB1 591-599 | SEQ ID NO: 10 | HLA-A*01:01, | 97.9% | 95.8% | 92% | |
| | (VSDGGPNLY) | HLA-A*29:02. | | | | |
| | | HLA-A*30:02, | | | | |
| | | HLA-A*80:01, | | | | |
| | | HLA-B*15:01, | | | | |
| | | HLA-B*15:17, | | | | |
| | | HLA-B*58:01, | | | | |
| | | HLA-C*08:02 | | | | |
| PA 282-290 | SEQ ID NO: 20 | HLA-A*02:01, | 93.9% | 96.7% | 98% | |
| | (FLLMDALKL) | HLA-A*02:02, | | | | |
| | | HLA-A*02:03, | | | | |
| | | HLA-A*02:06, | | | | |
| | | HLA-A*02:11, | | | | |
| | | HLA-A*02:12, | | | | |
| | | HLA-A*69:01 | | | | |
| NP 380-388 | SEQ ID NO: 2 | HLA-B*08:01, | 56% | 94% | 72% | |
| | (ELRSRYWAI) | HLA-A*02:01, | | | | |
| | | HLA-A*69:01, | | | | |
| | | HLA-B*18:01, | | | | |
| | | HLA-B*27:02 | | | | |
| NP 383-391 | SEQ ID NO: 3 | HLA-B*27:05, | 55.9% | 92% | 72% | |
| | (SRYWAIRTR) | HLA-B*27:03, | | | | |
| | | HLA-B*27:04, | | | | |
| | | HLA-B*27:06, | | | | |
| | | HLA-B*27:02, | | | | |
| | | HLA-B*27:09 | | | | |

TABLE 8

| | | World population |
|---|---|---|
| No. of epitopes | Sequence of epitopes | coverage (%) |
| 5 | SEQ ID NO: 1 (ILRGSVAHK), SEQ ID NO: 2 (ELRSRYWAI), | 92.56 |
| | SEQ ID NO: 3 (SRYWAIRTR), SEQ ID NO: 4 (CTELKLSDY), | |
| | SEQ ID NO: 5 (GILGFVFTL) | |
| 10 | SEQ ID NO: 1 (ILRGSVAHK), SEQ ID NO: 2 (ELRSRYWAI), | 99.23 |
| | SEQ ID NO: 3 (SRYWAIRTR), SEQ ID NO: 4 (CTELKLSDY), | |
| | SEQ ID NO: 5 (GILGFVFTL), SEQ ID NO: 6 (SIIPSGPLK), | |
| | SEQ ID NO: 7 (ASCMGLIY), SEQ ID NO: 8 (FMYSDFHFI), | |
| | SEQ ID NO: 9 (FVRQCFNPM), SEQ ID NO: 10 | |
| | (VSDGGPNLY) | |
| 15 | SEQ ID NO: 1 (ILRGSVAHK), SEQ ID NO: 2 (ELRSRYWAI), | 99.51 |
| | SEQ ID NO: 3 (SRYWAIRTR), SEQ ID NO: 4 (CTELKLSDY), | |
| | SEQ ID NO: 5 (GILGFVFTL), SEQ ID NO: 6 (SIIPSGPLK), | |
| | SEQ ID NO: 7 (ASCMGLIY), SEQ ID NO: 8 (FMYSDFHFI), | |
| | SEQ ID NO: 9 (FVRQCFNPM), SEQ ID NO: 10 | |
| | (VSDGGPNLY), SEQ ID NO: 11 (FLKDVMESM), SEQ ID | |
| | NO: 12 (NMLSTVLGV), SEQ ID NO: 13 (MMMGMFNML), | |
| | SEQ ID NO: 14 (YSHGTGTGY), SEQ ID NO: 15 | |
| | (HSNLNDATY) | |
| 20 | SEQ ID NO: 1 (ILRGSVAHK), SEQ ID NO: 2 (ELRSRYWAI), | 99.51 |
| | SEQ ID NO: 3 (SRYWAIRTR), SEQ ID NO: 4 (CTELKLSDY), | |
| | SEQ ID NO: 5 (GILGFVFTL), SEQ ID NO: 6 (SIIPSGPLK), | |
| | SEQ ID NO: 7 (ASCMGLIY), SEQ ID NO: 8 (FMYSDFHFI), | |
| | SEQ ID NO: 9 (FVRQCFNPM), SEQ ID NO: 10 | |
| | (VSDGGPNLY), SEQ ID NO: 11 (FLKDVMESM), SEQ ID | |
| | NO: 12 (NMLSTVLGV), SEQ ID NO: 13 (MMMGMFNML), | |
| | SEQ ID NO: 14 (YSHGTGTGY), SEQ ID NO: 15 | |
| | (HSNLNDATY), SEQ ID NO:16 (RRSGAAGAAVK), SEQ | |
| | ID NO: 17 (LLTEVETYV), SEQ ID NO: 18 (MVLASTTAK), | |
| | SEQ ID NO: 19 (RGINDRNFW), SEQ ID NO: 20 | |
| | (FLLMDALKL) | |

Here 15 epitopes give the same amount of coverage as 20 epitopes. However, to have more epitopes with the same HLA improves T cell induction, which is also necessary for good protection.

TABLE 9
Comparative analysis of the currently provided polyepitope
collection with the concept collection of Sharma et al.

| Criteria | Current TiHo work | Sharma et. al. |
|---|---|---|
| Sequences | Complete IAV database | n = 50 (4 pandemic strain, 46 seasonal |
| | | strain of IAV) |
| Seq. conservation | Human, avian, swine | Not added as criterion or |
| | Considered >50% conservation | characteristic |
| IAV proteins considered | NP, M1, PA, PB1 | NP, M1, HA (*HA is prone to |
| | | mutation and does barely not elicit |
| | | CTL after wild-virus infection) |
| CTL epitope database | IEDB | NetCTL 1.2 |
| Selection criterion | Peptides are all antigenic, and all | Not added as criterion or |
| | induce IFNγ by CD8+ T cells | characteristic |
| HLA coverage | 99.51% | 99% |
| Spacer seq. | AAY | AAY |
| Characteristics | In vitro immunogenicity data | Completely in silico data |
| | available | |
| | In vivo immunogenicity data | |
| | available | |
| Presentation as expressed protein | MVA | No results obtained |
| HTL | Not added as criterion or | Yes |
| | characteristic for immunogenicity | |
| PBL | Not added as criterion or | Yes |
| | characteristic for immunogenicity | |

Minimal Requirements for Optimal Liberation of Antigenic Peptides from Polyepitope Constructs

One of the most promising approaches of rational vaccine design uses so-called epitope-based vaccines (EVs). Vaccines based on T-cell epitopes, short immunogenic peptide sequences derived from antigens, offer several advantages over traditional whole attenuated or subunit vaccines. Unlike traditional vaccines, EVs do not contain potentially infectious material and the selection of peptides can be tailored to address the genetic variation of pathogens and that of a target population or of an individual patient. Well-established techniques for peptide synthesis guarantee rapid high-quality production and an economical storage of the final vaccine. To improve the recovery of epitopes, several groups have suggested the use of spacer sequences between epitopes. For each of the epitopes in the polyepitope construct to be immunogenic, the peptides need to be liberated by antigen processing efficiently for subsequent presentation to T cells. Thus, optimal cleavage of the polyepitope proteins by the proteasome is of utmost importance. It has been shown that presentation of epitopes may be dependent on the order of the peptides within the polyepitope protein, which dictated cleavage probability when such epitopes are not correctly spaced. The success of a polypeptide relies on efficient processing: constituent epitopes need to be recovered while avoiding neo-epitopes from epitope junctions. Spacers between epitopes are employed to ensure this, but spacer selection is non-trivial. Schubert and Kohlbacher present a framework to determine optimally the length and sequence of a spacer through multi-objective optimization for human leukocyte antigen class I restricted polypeptides. The method yields string-of-bead vaccines with flexible spacer lengths that increase the predicted epitope recovery rate fivefold while reducing the immunogenicity from neo-epitopes by 44% compared to designs without spacers. Therewith, spacer sequences adjacent to the epitopes can facilitate correct cleavage of the epitopes and prevent the creation of neo-epitopes, which can reduce vaccine efficacy (Schubert and Kohlbacher 2016). Here, the spacer sequence AAY (Ismail, Ahmad, and Azam 2020) was selected and beyond that, adding a degron to the polyepitope peptide of the disclosure was chosen, thereby avoiding the dependence on the order of the peptides within the polyepitope protein, which may dictate cleavage probability, generate a strong increase in the number of correctly cleaved epitopes and therewith a decrease in the neo-immunogenicity of the complete construct and have therewith disclosed independent recovery of individual epitopes irrespective of the order in which they are located in the polyepitope construct. Indeed, in experiments with expression plasmids (pCAGGS) that express three different epitopes (M1 58-66, NP 383-391 and NP 418-426) in all possible orders, differences were not observed in T cell activation when M1 58-66 peptide-specific T cells were co-cultured with HLA-A*0201 transgenic A549 cells transfected with the respective plasmids as measured by IFN-g ELISpot. Furthermore, with the MVA-PE construct, good antigenicity for four different epitopes that were located at positions 4, 5, 10 and 14 in the poly-epitope sequence was observed. This again demonstrates that the respective peptides are liberated from the poly-epitope sequence efficiently, independent of the position in the poly-epitope sequence.

### Enrichment of Peptide Specific CD8+ T Cells

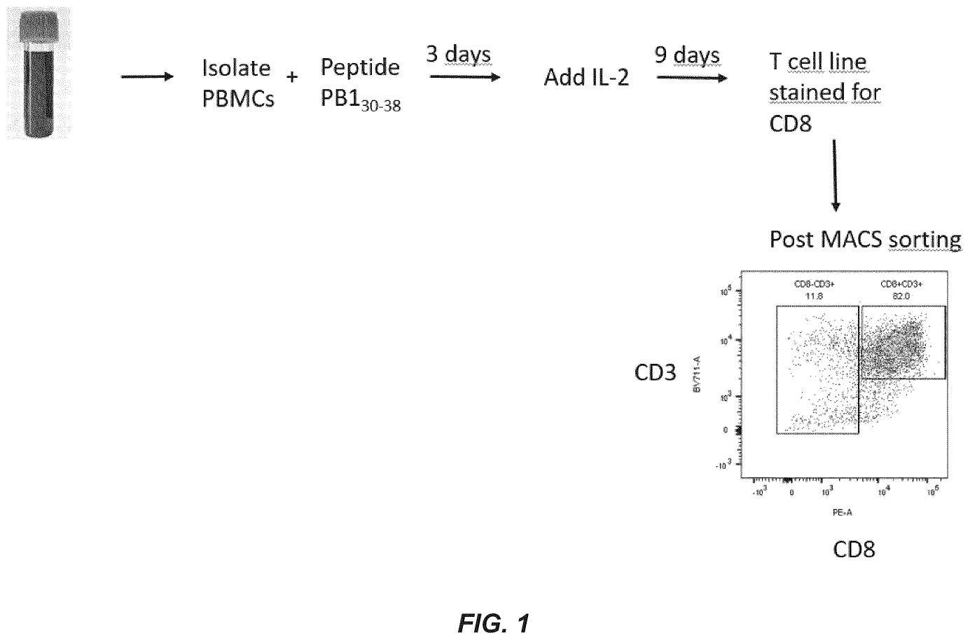

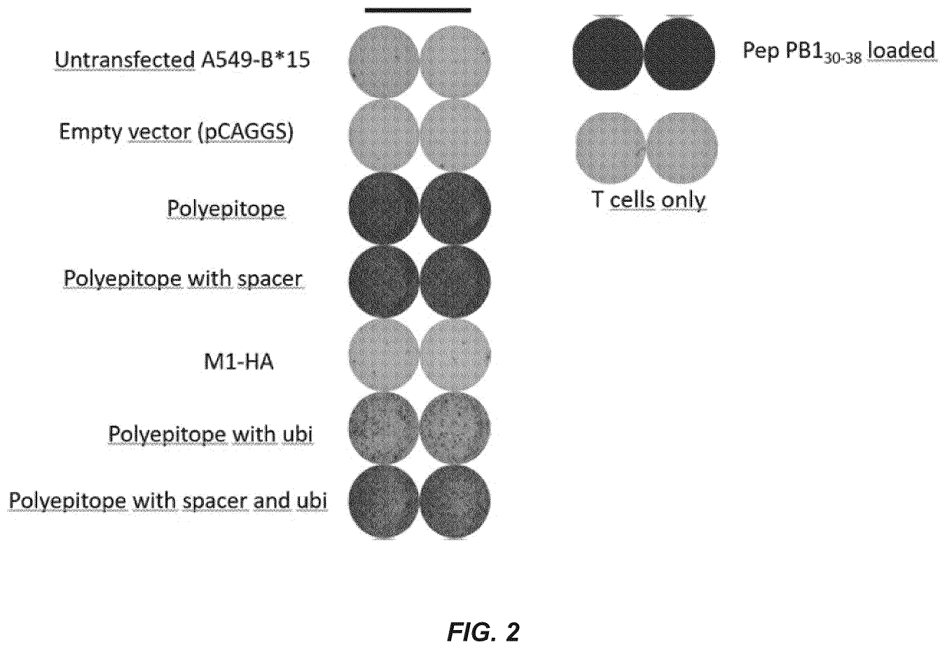

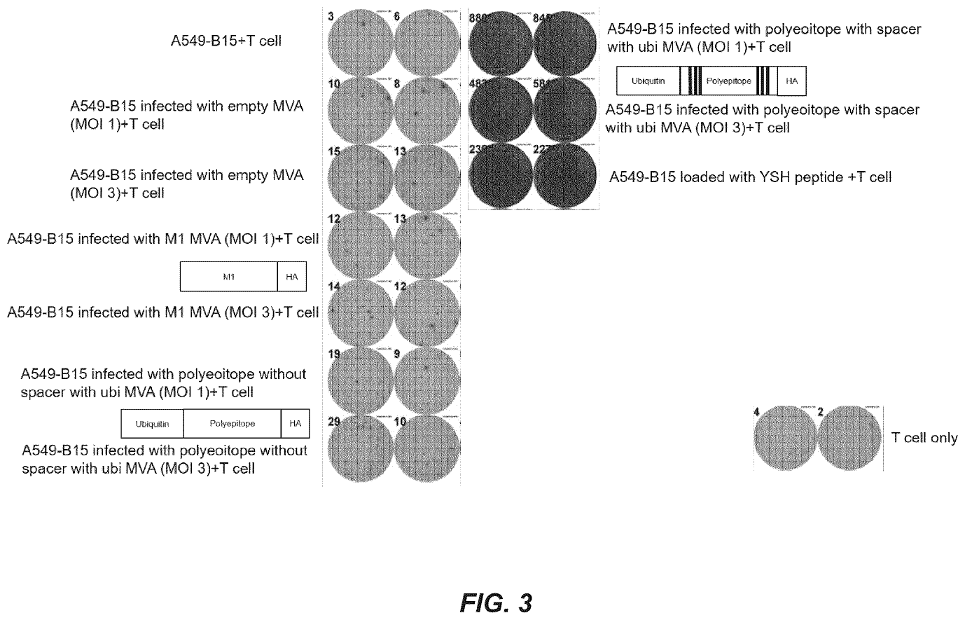

Epitope specific T cells are expanded using PBMCs from healthy HLA matched blood donors after stimulation with the corresponding peptide. After 12 days of expansion in vitro, enriched CD8+ T cells are isolated using magnetic beads coated with anti-CD8 antibody by using Magnetic-activated cell sorting (MACS) see FIG. 1.

In Vitro Antigenicity of Polyepitope Constructs in pCAGGS

Before embarking on generating recombinant MVAs that drive the expression of polyepitope sequences, addressing the issue of order of epitopes, spacer sequence and optimizing antigen processing by fusion of the sequence to ubiquitin is desired.

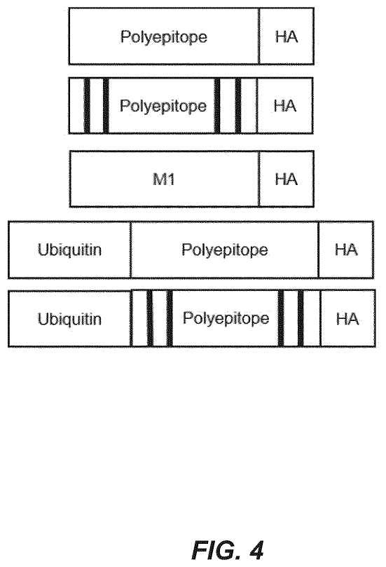

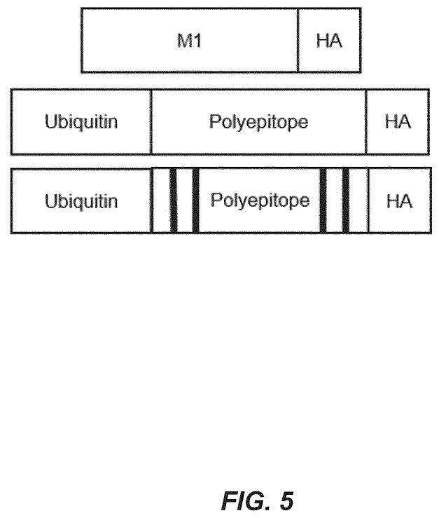

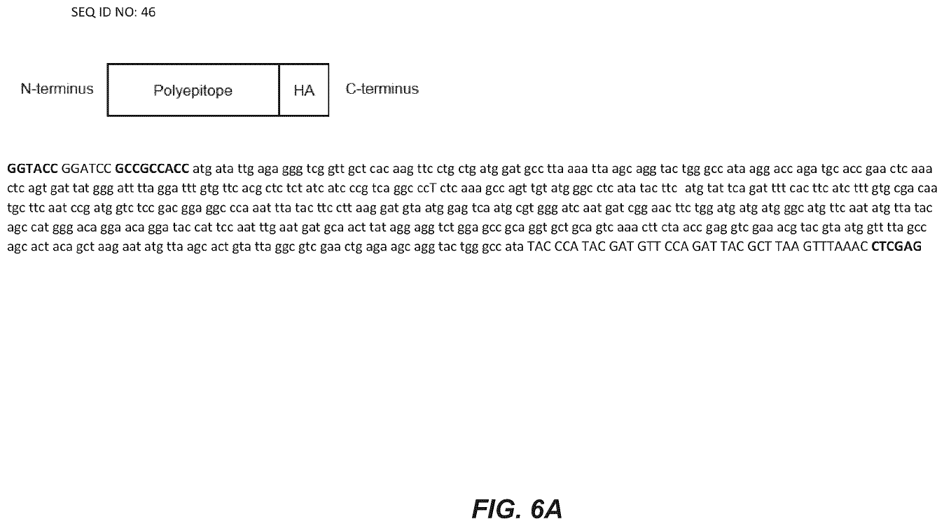

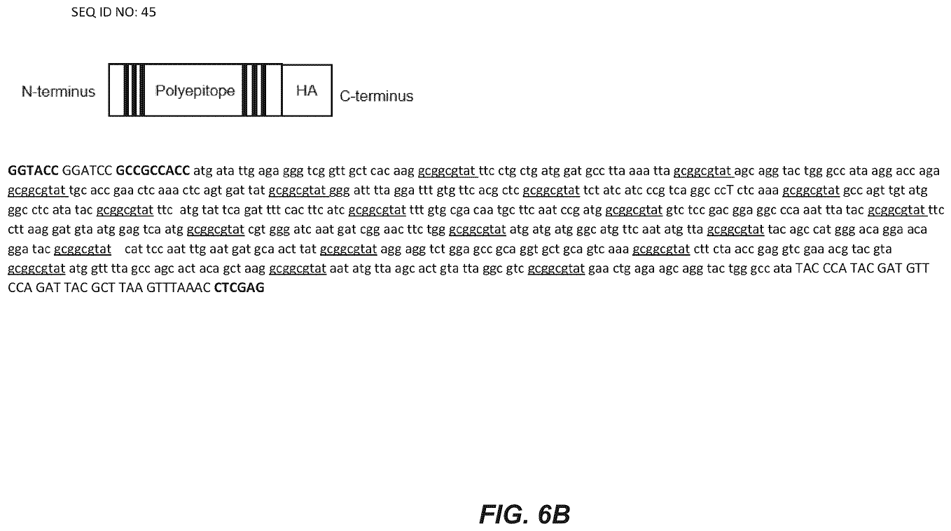

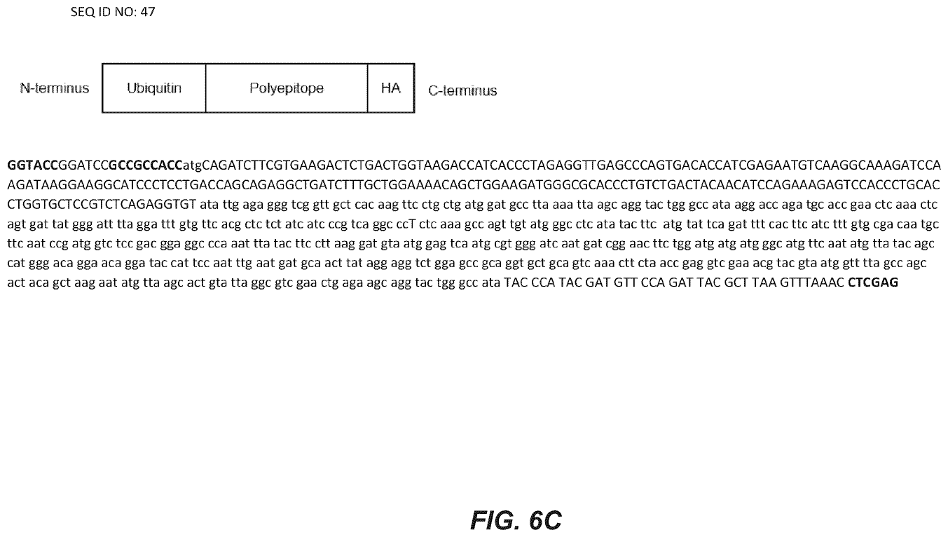

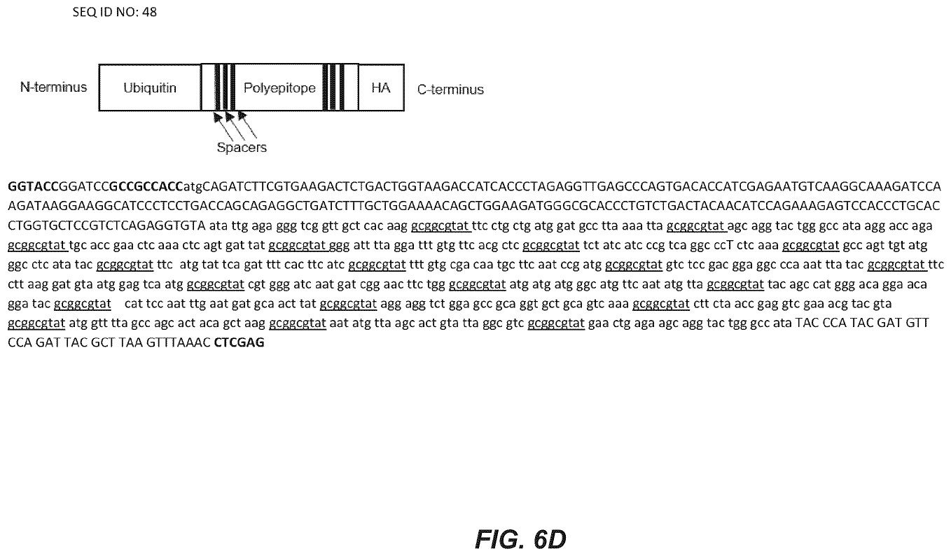

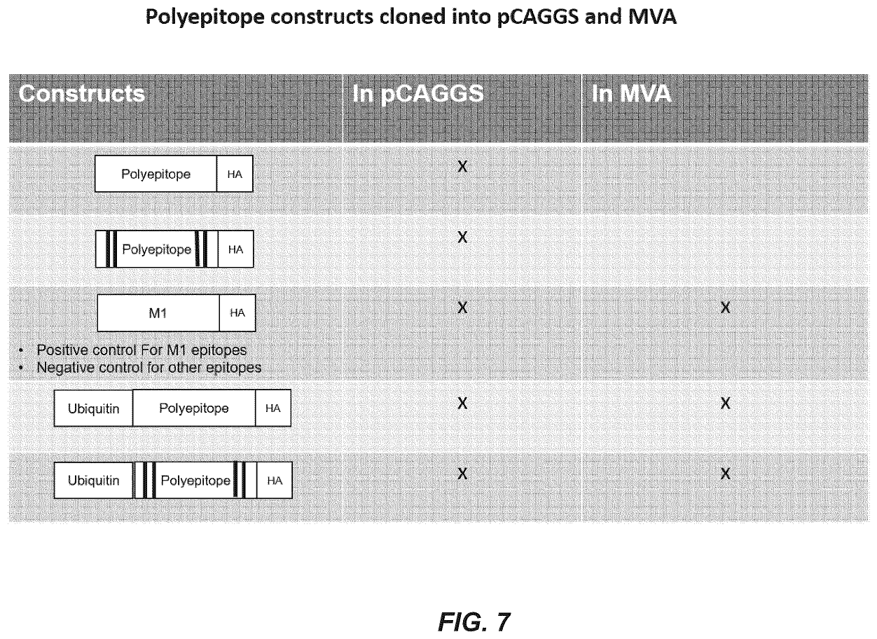

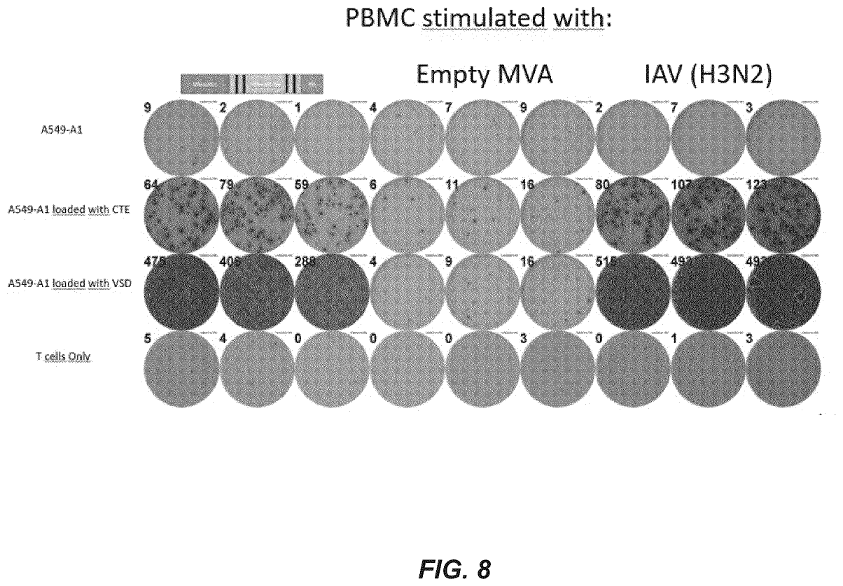

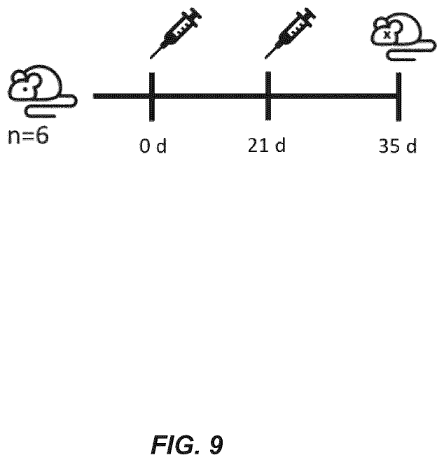

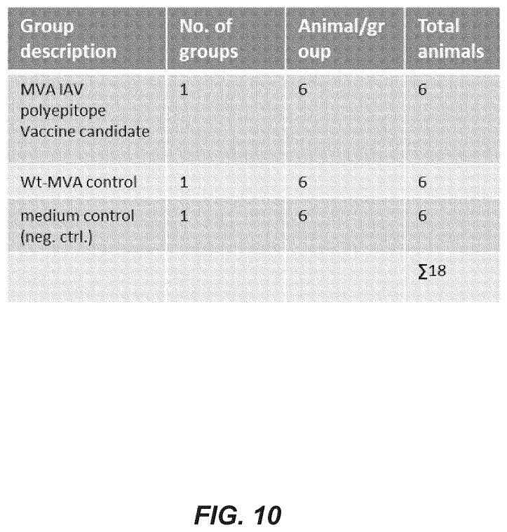

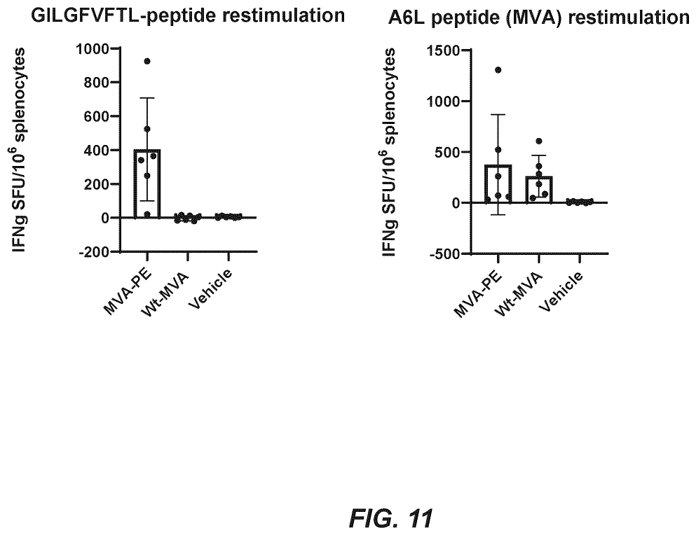

The 20 selected epitopes (from the table) are used to construct an artificial gene encoding the polyepitope sequence. Four different constructs are made with or without spacer (AAY) (supporting the liberation of the epitopes) and with or without ubiquitin (for docking the polyepitope to the proteasome for optimal antigen processing) as shown in the table below. In addition, a MVA-M1 construct is generated as a positive control for the Matrix epitopes, including the prototypic M 58-66 epitope. An HA-tag (SEQ ID NO:21 (YPYDVPDYA) is added to the C-Terminus of the polyepitope sequence for monitoring the expression of the target genes. These constructs are cloned in expression vector pCAGGS (Czudai-Matwich, Schnare, and Pinkenburg 2013). Successfully cloning all the polyepitope sequences in Pcaggs was accomplished. Constructs are shown in FIG. 4.

Transgenic A549 cells that constitutively express corresponding HLA genes have been generated previously using a retroviral system. HLA-transgenic A549 cells are transfected with expression plasmids (pCAGGS) encoding the respective artificial genes (polyepitope) or incubated with synthetic peptides corresponding to the single epitope contained in the polyepitope. Isolated CD8+ T cells (shown in FIG. 1) are co-cultured with transfected, and/or peptide loaded HLA transgenic A549 and IFN-γ secretion in response to peptide specific T-cell activation is measured by ELISpot. With the pCAGGS expression plasmids, an IFN-γ response in the enriched T cell populations in some of epitopes was observed, indicating that the epitopes could be liberated from the polyepitope construct and presented to specific T cells.

Similar results have been obtained for M1₅₈₋₆₆, NP₄₄₋₅₂, PB1₅₉₁₋₅₉₉ and PB1₃₀₋₃₈ epitopes, which are located on the 4^{th}, 5^{th}, 10^{th}, and 14^{th} position in the current expressed 20-epitope construct. All four epitopes are liberated from the polyepitope string and presented to specific T cells, ultimately leading to CD8+ T cell activation.

### In Vitro Antigenicity of Recombinant MVA Carrying Polyepitope

Similar polyepitope constructs (as shown in pCAGGS) are used to prepare recombinant MVAs. These artificial genes are cloned by using standard technique into a MVA shuttle vector, which facilitates homologous recombination and transient expression of a reporter gene (mCherry) for the selection of the recombinant MVAs. In this process, the constructs with ubiquitin and with or without spacer and rMVA-M1 could only be achieved. In vitro, evaluation of these constructs regarding their replication competence, genetic stability and antigenicity are further tested.

HLA-transgenic A549 cells are infected with recombinant MVAs expressing the polyepitope with ubiquitin (with or without spacer) or incubated with synthetic peptides corresponding to the single epitope contained in the polyepitope. Isolated CD8+ T cells (shown in FIG. 1) are co-cultured with infected, or peptide loaded HLA transgenic A549 and IFN-γ secretion in response to peptide specific T-cell activation is measured by ELISpot. Infection is done in two different MOIs (1 and 3). With the recombinant MVAs, an IFN-γ response in the enriched T cell populations in some of epitopes was observed, but typically only in the construct with spacer and ubiquitin, indicating that the epitopes could be liberated from the polyepitope construct and presented to specific T cells and the spacer have an important role in the proper liberation of single epitope from a polyepitope construct.

Similar results have been obtained for M1₅₈₋₆₆, NP₄₄₋₅₂, PB1₅₉₁₋₅₉₉ and PB1₃₀₋₃₈ epitopes, which are located on the 4^{th}, 5^{th}, 10^{th}, and 14^{th} position in the current polyepitope construct. All epitopes are liberated from the polyepitope string (with spacer and ubiquitin) and presented to specific T cells, ultimately leading to CD8+ T cell activation.

### In Vitro Immunogenicity

It was desired to test the in vitro immunogenicity of the rMVA-construct that express the synthetic gene (SEQ ID NO:48) encoding the polyepitope sequence with spacers fused to ubiquitin (rMVA-PE). To this end, PBMCs of an HLA-A*01 positive healthy blood donor were stimulated with rMVA-PE or wild type MVA (negative control) or IAV (influenza A virus positive control). After expansion of specific T cells, the presence of influenza A virus epitope specific T cells was assessed by IFNγ Elispot assay after restimulation of the expanded T cells with two HLA-A*01-restricted peptides, SEQ ID NO:4 (CTELKLSDY) (NP 44-52) and SEQ ID NO:10 (VSDGGPNLY) (PB1₅₉₁₋₅₉₉). The magnitude of the in vitro response induced by rMVA-PE was like that induced with IAV. Furthermore, the immunodominance hierarchy between the two peptides tested was also similar. It was concluded that with rMVA-PE, a T cell response was induced that represented the T cell response induced by stimulation with IAV, in vitro.

### In Vivo Immunogenicity

Groups of 6-8-week-old female C57BL/6 tg HLA A*02:01 mice (n=6) were immunized twice intramuscularly (days 0 and 21) with 10⁷ PFU of rMVA-PE, wt MVA, or with buffer (See FIGS. 9 and 10). Fourteen days after the last immunization, the mice were sacrificed and their spleens were harvested and processed to obtain single-cell suspensions using the gentleMACS Octo Dissociator (Miltenyi Biotec, Bergisch Gladbach, Germany) and passed through 100 μm and 70 μm cell strainers (Miltenyi Biotec, Bergisch Gladbach, Germany). Erythrocytes were lysed in ACK lysing buffer (Gibco, Waltham, MA, USA) for 1.5 min at RT, followed by washing with cold PBS containing 2% FBS. Subsequently, splenocytes were resuspended in RPMI 1640 (Gibco, Waltham, MA, USA) supplemented with 10% FBS, 10 mM HEPES and 1% P/S (R10F) and kept on ice until further use. Splenocytes (2.5×10⁵ cells per well) were incubated in triplicate either with individual HLA-A*02:01 restricted peptides (10 μM) from the IAV M1 protein SEQ ID NO:5 (GILGFVFTL, M1₅₈₋₆₆) or from MVA (A6L) in pre-coated 96-well plates (Mouse IFN-ELISpotPLUS kit, Mabtech, Nacka Strand, Sweden) for 30 h at 37° C. Cells incubated with PMA/ionomycin (both Cayman Chemical Company, Ann Arbor, MI, USA) or DMSO were used as positive and negative controls, respectively. Plates were developed according to the manufacturer's instructions. Developed plates were scanned using an ImmunoSpot S6 Ultimate M2 reader and spots were counted using the ImmunoSpot software version 7.0.9.5 (both Cellular Technology Limited, Shaker Heights, OH, USA). Data are presented as mean SFU per 10⁶ splenocytes after subtraction of the negative control.

rMVA-PE vaccinated mice displayed a response to the IAV M1₅₈₋₆₆ epitope that was not observed in wt-MVA immunized or mock-immunized mice. Both rMVA-PE and wt MVA mounted a response to the MVA-derived A6L epitope. As the available experimental HLA-repertoire of transgenic mice is now depleted in respect to other epitopes listed herein, human phase I trials are now being planned and discussed with authorities to provide for accreditation of testing the broad HLA-restricted immune response elicited by rMVA-PE in clinical trials in humans.

Sequences listed

| CD8+ Epitope | Sequence | Nucleotide |
|---|---|---|
| NP ₂₆₅₋₂₇₄ | SEQ ID NO: 1 | SEQ ID NO: 22 (ata ttg aga ggg tcg |
| | (ILRGSVAHK) | gtt gct cac aag) |
| NP ₃₈₀₋₃₈₈ | SEQ ID NO: 2 | SEQ ID NO: 23 (gaa ctg aga agc |
| | (ELRSRYWAI) | agg tac tgg gcc ata) |
| NP ₃₈₃₋₃₉₁ | SEQ ID NO: 3 | SEQ ID NO: 24 (agc agg tac tgg |
| | (SRYWAIRTR) | gcc ata agg acc aga) |
| NP ₄₄₋₅₂ | SEQ ID NO: 4 | SEQ ID NO: 25 (tgc acc gaa ctc |
| | (CTELKLSDY) | aaa ctc agt gat tat) |
| M1 ₅₈₋₆₆ | SEQ ID NO: 5 | SEQ ID NO: 26 (ggg att tta gga ttt |
| | (GILGFVFTL) | gtg ttc acg ctc) |
| M1 ₁₃₋₂₁ | SEQ ID NO: 6 | SEQ ID NO: 27 (tct atc atc ccg tca |
| | (SIIPSGPLK) | ggc ccc ctc aaa) |
| M1 ₁₂₈₋₁₃₅ | SEQ ID NO: 7 | SEQ ID NO: 28 (gcc agt tgt atg ggc |
| | (ASCMGLIY) | ctc ata tac) |
| PA ₄₆₋₅₁ | SEQ ID NO: 8 | SEQ ID NO: 29 (ttc atg tat tca gat |
| | (FMYSDFHFI) | ttt cac ttc atc) |
| PA ₄₋₁₂ | SEQ ID NO: 9 | SEQ ID NO: 30 (ttt gtg cga caa tgc |
| | (FVRQCFNPM) | ttc aat ccg atg) |
| PB1 ₅₉₁₋₅₉₉ | SEQ ID NO: 10 | SEQ ID NO: 31 (gtc tcc gac gga |
| | (VSDGGPNLY) | ggc cca aat tta tac) |
| PB1 ₁₆₆₋₁₇₄ | SEQ ID NO: 11 | SEQ ID NO: 32 (ttc ctt aag gat gta |
| | (FLKDVMESM) | atg gag tca atg) |
| PB1 ₄₁₃₋₄₂₁ | SEQ ID NO: 12 | SEQ ID NO: 33 (aat atg tta agc act |
| | (NMLSTVLGV) | gta tta ggc gtc) |
| PB1 ₄₀₇₋₄₁₅ | SEQ ID NO: 13 | SEQ ID NO: 34 (atg atg atg ggc atg |
| | (MMMGMFNML) | ttc aat atg tta) |
| PB1 ₃₀₋₃₈ | SEQ ID NO: 14 | SEQ ID NO: 35 (tac agc cat ggg |
| | (YSHGTGTGY) | aca gga aca gga tac) |
| NP ₁₄₀₋₁₄₈ | SEQ ID NO: 15 | SEQ ID NO: 36 (cat tcc aat ttg aat |
| | (HSNLNDATY) | gat gca act tat) |
| NP ₁₇₄₋₁₈₄ | SEQ ID NO: 16 | SEQ ID NO: 37 (agg agg tct gga |
| | (RRSGAAGAAVK) | gcc gca ggt gct gca gtc aaa) |
| M1 ₃₋₁₁ | SEQ ID NO: 17 | SEQ ID NO: 38 (ctt cta acc gag gtc |
| | (LLTEVETYV) | gaa acg tac gta |
| M1 ₁₇₉₋₁₈₇ | SEQ ID NO: 18 | SEQ ID NO: 39 (atg gtt tta gcc agc |
| | (MVLASTTAK) | act aca gct aag) |
| NP ₁₉₉₋₂₀₇ | SEQ ID NO: 19 | SEQ ID NO: 40 (cgt ggg atc aat gat |
| | (RGINDRNFW) | cgg aac ttc tgg |
| PA ₂₈₂₋₂₉₀ | SEQ ID NO: 20 | SEQ ID NO: 41 (ttc ctg ctg atg gat |
| | (FLLMDALKL) | gcc tta aaa tta) |

Start Codon: atg
Stop Codon: TAA
BamH1: GGATCC
Kozak: GCCGCCACC
Pme1 : GTTTAAAC
KpnI: GGTACC (for pCAGGS)
XhoI: CTCGAG (for pCAGGS)
HA tag SEQ ID NO: 21 (YPYDVPDYA): SEQ ID NO: 42 (TACCCATACGAT
GTTCCAGATTACGCT)
Spacer AAY: gcggcgtat
Ubiquitin: SEQ ID NO: 43
(CAGATCTTCGTGAAGACTCTGACTGGTAAGACCATCACCCTCGAGGTTGAGCCCAGTGACACCATCGAG
AATGTCAAGGCAAAGATCCAAGATAAGGAAGGCATCCCTCCTGACCAGCAGAGGCTGATCTTTGCTGGAA
AACAGCTGGAAGATGGGCGCACCCTGTCTGACTACAACATCCAGAAAGAGTCCACCCTGCACCTGGTGCT
CCGTCTCAGAGGTGTA)
Optimized M1-HA (809 bp)
SEQ ID NO: 44 (GGTACC GGATCCGCCGCCACC atg agt ctt cta acc gag gtc gaa acg
tac gta ctc tct atc atc ccg tca ggc ccT ctc aaa gcc gag atc gca cag aga
ctt gaa gat gtc ttt gca ggg aag aac acc gat ctt gag gtt ctc atg gaa tgg
cta aag aca aga cca atc ctg tca cct ctg act aag ggT att tta gga ttt gtg
ttc acg ctc acc gtg ccc agt gag cga gga ctg cag cgt aga cgc ttt gtc caa
aat gcc ctt aat ggg aac ggT gat cca aat aac atg gac aaa gca gtt aaa ctg
tat agg aag ctc aag agg gag ata aca ttc cat ggT gcc aaa gaa atc tca ctc
agt tat tct gct ggt gca ctt gcc agt tgt atg ggc ctc ata tac aac agg atg
ggT gct gtg acc act gaa gtg gca ttt ggc ctg gta tgt gca acc tgt gaa cag
att gct gac tcc cag cat cgg tct cat agg caa atg gtg aca aca acc aat cca
cta atc aga cat gag aac aga atg gtt tta gcc agc act aca gct aag gct atg
gag caa atg gct gga tcg agt gag caa gca gca gag gcc atg gag gtt gct agt
cag gct aga caa atg gtg caa gcg atg aga acc att ggg act cat cct agc tcc
agt gct ggt ctg aaa aat gat ctt ctt gaa aat ttg cag gcc tat cag aaa cga
atg ggT gtg cag atg caa cgg ttc aag TAC CCA TAC GAT GTT CCA GAT TAC GCT
TAA GTTTAAAC CTCGAG)
Optimized Polyepitope with spacer: (770 bp)
SEQ ID NO: 45 (GGTACC GGATCC GCCGCCACC atg ata ttg aga ggg tcg gtt gct
cac aag gcggcgtat ttc ctg ctg atg gat gcc tta aaa tta gcggcgtat agc agg
tac tgg gcc ata agg acc aga gcggcgtat tgc acc gaa ctc aaa ctc agt gat
tat gcggcgtat ggg att tta gga ttt gtg ttc acg ctc gcggcgtat tct atc atc
ccg tca ggc ccT ctc aaa gcggcgtat gcc agt tgt atg ggc ctc ata tac
gcggcgtat ttc atg tat tca gat ttt cac ttc atc gcggcgtat ttt gtg cga caa
tgc ttc aat ccg atg gcggcgtat gtc tcc gac gga ggc cca aat tta tac
gcggcgtat ttc ctt aag gat gta atg gag tca atg gcggcgtat cgt ggg atc aat
gat cgg aac ttc tgg gcggcgtat atg atg atg ggc atg ttc aat atg tta
gcggcgtat tac agc cat ggg aca gga aca gga tac gcggcgtat cat tcc aat
ttg aat gat gca act tat gcggcgtat agg agg tct gga gcc gca ggt gct gca
gtc aaa gcggcgtat ctt cta acc gag gtc gaa acg tac gta gcggcgtat atg gtt
tta gcc agc act aca gct aag gcggcgtat aat atg tta agc act gta tta ggc
gtc gcggcgtat gaa ctg aga agc agg tac tgg gcc ata TAC CCA TAC GAT GTT
CCA GAT TAC GCT TAA GTTTAAAC CTCGAG)
Optimized Polyepitope without spacer (599 bp)
SEQ ID NO: 46 (GGTACC GGATCC GCCGCCACC atg ata ttg aga ggg tcg gtt gct
cac aag ttc ctg ctg atg gat gcc tta aaa tta agc agg tac tgg gcc ata agg
acc aga tgc acc gaa ctc aaa ctc agt gat tat ggg att tta gga ttt gtg ttc
acg ctc tct atc atc ccg tca ggc ccT ctc aaa gcc agt tgt atg ggc ctc ata
tac ttc atg tat tca gat ttt cac ttc atc ttt gtg cga caa tgc ttc aat ccg
atg gtc tcc gac gga ggc cca aat tta tac ttc ctt aag gat gta atg gag tca
atg cgt ggg atc aat gat cgg aac ttc tgg atg atg atg ggc atg ttc aat atg
tta tac agc cat ggg aca gga aca gga tac cat tcc aat ttg aat gat gca act
tat agg agg tct gga gcc gca ggt gct gca gtc aaa ctt cta acc gag gtc gaa
acg tac gta atg gtt tta gcc agc act aca gct aag aat atg tta agc act gta
tta ggc gtc gaa ctg aga agc agg tac tgg gcc ata TAC CCA TAC GAT GTT CCA
GAT TAC GCT TAA GTTTAAAC CTCGAG)
Optimized Polyepitope without spacer with ubiquitin (823 bp)
SEQ ID NO: 47
(GGTACCGGATCCGCCGCCACCatgCAGATCTTCGTGAAGACTCTGACTGGTAAGACCATCACCCTAGAG
GTTGAGCCCAGTGACACCATCGAGAATGTCAAGGCAAAGATCCAAGATAAGGAAGGCATCCCTCCTGACC
AGCAGAGGCTGATCTTTGCTGGAAAACAGCTGGAAGATGGGCGCACCCTGTCTGACTACAACATCCAGAA
AGAGTCCACCCTGCACCTGGTGCTCCGTCTCAGAGGTGT ata ttg aga ggg tcg gtt gct cac
aag ttc ctg ctg atg gat gcc tta aaa tta agc agg tac tgg gcc ata agg acc
aga tgc acc gaa ctc aaa ctc agt gat tat ggg att tta gga ttt gtg ttc acg
ctc tct atc atc ccg tca ggc ccT ctc aaa gcc agt tgt atg ggc ctc ata tac
ttc atg tat tca gat ttt cac ttc atc ttt gtg cga caa tgc ttc aat ccg atg
gtc tcc gac gga ggc cca aat tta tac ttc ctt aag gat gta atg gag tca atg
cgt ggg atc aat gat cgg aac ttc tgg atg atg atg ggc atg ttc aat atg tta
tac agc cat ggg aca gga aca gga tac cat tcc aat ttg aat gat gca act tat
agg agg tct gga gcc gca ggt gct gca gtc aaa ctt cta acc gag gtc gaa acg
tac gta atg gtt tta gcc agc act aca gct aag aat atg tta agc act gta tta
ggc gtc gaa ctg aga agc agg tac tgg gcc ata TAC CCA TAC GAT GTT CCA GAT
TAC GCT TAA GTTTAAAC CTCGAG)
Optimized Polyepitope with spacer and ubiquitin (995 bp)
SEQ ID NO: 48
(GGTACCGGATCCGCCGCCACCatgCAGATCTTCGTGAAGACTCTGACTGGTAAGACCATCACCCTAGAG
GTTGAGCCCAGTGACACCATCGAGAATGTCAAGGCAAAGATCCAAGATAAGGAAGGCATCCCTCCTGACC
AGCAGAGGCTGATCTTTGCTGGAAAACAGCTGGAAGATGGGCGCACCCTGTCTGACTACAACATCCAGAA
AGAGTCCACCCTGCACCTGGTGCTCCGTCTCAGAGGTGTA ata ttg aga ggg tcg gtt gct cac
aag gcggcgtat ttc ctg ctg atg gat gcc tta aaa tta gcggcgtat agc agg tac
tgg gcc ata agg acc aga gcggcgtat tgc acc gaa ctc aaa ctc agt gat tat
gcggcgtat ggg att tta gga ttt gtg ttc acg ctc gcggcgtat tct atc atc ccg
tca ggc ccT ctc aaa gcggcgtat gcc agt tgt atg ggc ctc ata tac gcggcgtat
ttc atg tat tca gat ttt cac ttc atc gcggcgtat ttt gtg cga caa tgc ttc
aat ccg atg gcggcgtat gtc tcc gac gga ggc cca aat tta tac gcggcgtat ttc
ctt aag gat gta atg gag tca atg gcggcgtat cgt ggg atc aat gat cgg aac
ttc tgg gggcgtat atg atg atg ggc atg ttc aat atg tta gcggcgtat tac agc
cat ggg aca gga aca gga tac gcggcgtat cat tcc aat ttg aat gat gca act
tat gcggcgtat agg agg tct gga gcc gca ggt gct gca gtc aaa gcggcgtat ctt
cta acc gag gtc gaa acg tac gta gcggcgtat atg gtt tta gcc agc act aca
gct aag gcggcgtat aat atag tta agc act gta tta ggc gtc gcggcgtat gaa ctg
aga agc agg tac tgg gcc ata TAC CCA TAC GAT GTT CCA GAT TAC GCT TAA
GTTTAAAC CTCGAG)

### REFERENCES

‘Antiviral drugs in influenza: an adjunct to vaccination in some situations’. 2006. Prescrire Int, 15: 21-30.
Atsmon, J., E. Kate-Ilovitz, D. Shaikevich, Y. Singer, I. Volokhov, K. Y. Haim, and T. Ben-Yedidia. 2012. ‘Safety and immunogenicity of multimeric-001—a novel universal influenza vaccine’, J Clin Immunol, 32: 595-603.
Bodmer, J. G., S. G. Marsh, E. D. Albert, W. F. Bodmer, R. E. Bontrop, B. Dupont, H. A. Erlich, J. A. Hansen, B. Mach, W. R. Mayr, P. Parham, E. W. Petersdorf, T. Sasazuki, G. M. Schreuder, J. L. Strominger, A. Svejgaard, and P. I. Terasaki. 1999. ‘Nomenclature for factors of the HLA system, 1998’, Vox Sang, 77: 164-91.
Boon, A. C., G. de Mutsert, R. A. Fouchier, A. D. Osterhaus, and G. F. Rimmelzwaan. 2006. ‘The hypervariable immunodominant NP418-426 epitope from the influenza A virus nucleoprotein is recognized by cytotoxic T lymphocytes with high functional avidity’, J Virol, 80: 6024-32.
Bui, H. H., J. Sidney, W. Li, N. Fusseder, and A. Sette. 2007. ‘Development of an epitope conservancy analysis tool to facilitate the design of epitope-based diagnostics and vaccines’, BMC Bioinformatics, 8: 361.
Corradin, G., H. M. Etlinger, and J. M. Chiller. 1977. ‘Lymphocyte specificity to protein antigens. I. Characterization of the antigen-induced in vitro T cell-dependent proliferative response with lymph node cells from primed mice’, J Immunol, 119: 1048-53.
Czudai-Matwich, V., M. Schnare, and O. Pinkenburg. 2013. ‘A simple and fast system for cloning influenza A virus gene segments into pHW2000- and pCAGGS-based vectors’, Arch Virol, 158: 2049-58.
de Vries, R. D., A. F. Altenburg, N. J. Nieuwkoop, E. de Bruin, S. E. van Trierum, M. R. Pronk, M. M. Lamers, M. Richard, D. F. Nieuwenhuijse, M. P. G. Koopmans, Jhcm Kreijtz, R. A. M. Fouchier, Adme Osterhaus, G. Sutter, and G. F. Rimmelzwaan. 2018. ‘Induction of Cross-Clade Antibody and T-Cell Responses by a Modified Vaccinia Virus Ankara-Based Influenza A(H5N1) Vaccine in a Randomized Phase 1/2a Clinical Trial’, J Infect Dis, 218: 614-23.
Effros, R. B., P. C. Doherty, W. Gerhard, and J. Bennink. 1977. ‘Generation of both cross-reactive and virus-specific T-cell populations after immunization with serologically distinct influenza A viruses’, J Exp Med, 145: 557-68.
Ershler, W. B., S. Gravenstein, and Z. S. Geloo. 2007. ‘Thymosin alpha 1 as an adjunct to influenza vaccination in the elderly: rationale and trial summaries’, Ann N Y Acad Sci, 1112: 375-84.
Evans, T. G., L. Bussey, E. Eagling-Vose, K. Rutkowski, C. Ellis, C. Argent, P. Griffin, J. Kim, S. Thackwray, S. Shakib, J. Doughty, J. Gillies, J. Wu, J. Druce, M. Pryor, and S. Gilbert. 2022. ‘Efficacy and safety of a universal influenza A vaccine (MVA-NP+M1) in adults when given after seasonal quadrivalent influenza vaccine immunization (FLU009): a phase 2b, randomized, double-blind trial’, Lancet Infect Dis.
Ismail, S., S. Ahmad, and S. S. Azam. 2020. ‘Immunoinformatics characterization of SARS-CoV-2 spike glycoprotein for prioritization of epitope based multivalent peptide vaccine’, J Mol Liq, 314: 113612.
Leggatt, G. R. 2014. ‘Peptide Dose and/or Structure in Vaccines as a Determinant of T Cell Responses’, Vaccines (Basel), 2: 537-48.
Leitner, W. W., H. Ying, and N. P. Restifo. 1999. ‘DNA and RNA-based vaccines: principles, progress and prospects’, Vaccine, 18: 765-77.
McElhaney, J. E. 2011. ‘Influenza vaccine responses in older adults’, Ageing Res Rev, 10: 379-88.
Pleguezuelos, O., E. James, A. Fernandez, V. Lopes, L. A. Rosas, A. Cervantes-Medina, J. Cleath, K. Edwards, D. Neitzey, W. Gu, S. Hunsberger, J. K. Taubenberger, G. Stoloff, and M. J. Memoli. 2020. ‘Efficacy of FLU-v, a broad-spectrum influenza vaccine, in a randomized phase IIb human influenza challenge study’, NPJ Vaccines, 5: 22.
Ravid, T., and M. Hochstrasser. 2008. ‘Diversity of degradation signals in the ubiquitin-proteasome system’, Nat Rev Mol Cell Biol, 9: 679-90.
Romeli, S., S. S. Hassan, and W. B. Yap. 2020. ‘Multi-Epitope Peptide-Based and Vaccinia-Based Universal Influenza Vaccine Candidates Subjected to Clinical Trials’, Malays J Med Sci, 27: 10-20.
Schubert, B., and O. Kohlbacher. 2016. ‘Designing string-of-beads vaccines with optimal spacers’, Genome Med, 8: 9.
Sharma, S., V. Kumari, B. V. Kumbhar, A. Mukherjee, R. Pandey, and K. Kondabagil. 2021. ‘Immunoinformatics approach for a novel multi-epitope subunit vaccine design against various subtypes of Influenza A virus’, Immunobiology, 226: 152053.
Song, F., R. Fux, L. B. Provacia, A. Volz, M. Eickmann, S. Becker, A. D. Osterhaus, B. L. Haagmans, and G. Sutter. 2013. ‘Middle East respiratory syndrome coronavirus spike protein delivered by modified vaccinia virus Ankara efficiently induces virus-neutralizing antibodies’, J Virol, 87: 11950-4.
Stawikowski, M., and G. B. Fields. 2012. ‘Introduction to peptide synthesis’, Curr Protoc Protein Sci, Chapter 18: Unit 18.1.
Stoloff, G. A., and W. Caparros-Wanderley. 2007. ‘Synthetic multi-epitope peptides identified in silico induce protective immunity against multiple influenza serotypes’, Eur J Immunol, 37: 2441-9.
Tregoning, J. S., R. F. Russell, and E. Kinnear. 2018. ‘Adjuvanted influenza vaccines’, Hum Vaccin Immunother, 14: 550-64.
van Doorn, E., H. Liu, T. Ben-Yedidia, S. Hassin, I. Visontai, S. Norley, H. W. Frijlink, and E. Hak. 2017. ‘Evaluating the immunogenicity and safety of a BiondVax-developed universal influenza vaccine (Multimeric-001) either as a standalone vaccine or as a primer to H5N1 influenza vaccine: Phase IIb study protocol’, Medicine (Baltimore), 96: e6339.
Zammit, D. J., L. S. Cauley, Q. M. Pham, and L. Lefrançois. 2005. ‘Dendritic cells maximize the memory CD8 T cell response to infection’, Immunity, 22: 561-70.

1.-16. (canceled)

17. A polynucleotide encoding at least a polyepitope polypeptide having at least five influenza A virus (IAV)-derived peptides that induce IFNγ by CD8+ T cells, the at least five IAV-derived peptides having SEQ ID NO:8 (FMYSDFHFI), SEQ ID NO:9 (FVRQCFNPM), SEQ ID NO:12 (NMLSTVLGV), SEQ ID NO:13 (MMMGMFNML), and SEQ ID NO:14 (YSHGTGTGY),
wherein the polynucleotide also has genetic information that provides peptide spacer (linker) placement adjacent to IAV-derived peptides, and
wherein the polynucleotide has a polynucleotide encoding a degron.

18. The polynucleotide of claim 17 encoding a further five IAV-derived peptides that induce IFNγ by CD8+ T cells, the further five peptides having SEQ ID NO:5 (GILGFVFTL), SEQ ID NO:3 (SRYWAIRTR), SEQ ID NO:16 (RRSGAAGAAVK), SEQ ID NO:2 (ELRSRYWAI), and SEQ ID NO:4 (CTELKLSDY).

19. The polynucleotide of claim 17 encoding at least 15 IAV-derived peptides that induce IFNγ by CD8+ T cells, the 15 IAV-derived peptides selected from the group consisting of SEQ ID NO:1 (ILRGSVAHK), SEQ ID NO:2 (ELRSRYWAI), SEQ ID NO:3 (SRYWAIRTR), SEQ ID NO:4 (CTELKLSDY), SEQ ID NO:5 (GILGFVFTL), SEQ ID NO:6 (SIIPSGPLK), SEQ ID NO:7 (ASCMGLIY), SEQ ID NO:8 (FMYSDFHFI), SEQ ID NO:9 (FVRQCFNPM), SEQ ID NO:10 (VSDGGPNLY), SEQ ID NO:11 (FLKDVMESM), SEQ ID NO:12 (NMLSTVLGV), SEQ ID NO:13 (MMMGMFNML), SEQ ID NO:14 (YSHGTGTGY), SEQ ID NO:15 (HSNLNDATY), SEQ ID NO:16 (RRSGAAGAAVK), SEQ ID NO:17 (LLTEVETYV), SEQ ID NO:18 (MVLASTTAK), SEQ ID NO:19 (RGINDRNFW), and SEQ ID NO:20 (FLLMDALKL).

20. The polynucleotide of claim 17 encoding 20 IAV-derived peptides that induce IFNγ by CD8+ T cells, with amino acid sequences SEQ ID NO:1 (ILRGSVAHK), SEQ ID NO:2 (ELRSRYWAI), SEQ ID NO:3 (SRYWAIRTR), SEQ ID NO:4 (CTELKLSDY), SEQ ID NO:5 (GILGFVFTL), SEQ ID NO:6 (SIIPSGPLK), SEQ ID NO:7 (ASCMGLIY), SEQ ID NO:8 (FMYSDFHFI), SEQ ID NO:9 (FVRQCFNPM), SEQ ID NO:10 (VSDGGPNLY), SEQ ID NO:11 (FLKDVMESM), SEQ ID NO:12 (NMLSTVLGV), SEQ ID NO:13 (MMMGMFNML), SEQ ID NO:14 (YSHGTGTGY), SEQ ID NO:15 (HSNLNDATY), SEQ ID NO:16 (RRSGAAGAAVK), SEQ ID NO:17 (LLTEVETYV), SEQ ID NO:18 (MVLASTTAK), SEQ ID NO:19 (RGINDRNFW), and SEQ ID NO:20 (FLLMDALKL).

21. The polynucleotide of claim 17, wherein the peptide spacer (linker) comprises tripeptide AAY.

22. The polynucleotide of claim 17, wherein the degron comprises ubiquitin.

23. A nucleic acid vector, virus, cell, or formulation comprising the polynucleotide of claim 17.

24. A proteinaceous formulation comprising a polyepitope polypeptide derived from the polynucleotide of claim 17.

25. A polyepitope polypeptide comprising:
at least five influenza A virus (IAV)-derived peptides that induce IFNγ by CD8+ T cells, wherein the at least five IAV-derived peptides are SEQ ID NO:8 (FMYSDFHFI), SEQ ID NO:9 (FVRQCFNPM), SEQ ID NO:12 (NMLSTVLGV), SEQ ID NO:13 (MMMGMFNML), and SEQ ID NO:14 (YSHGTGTGY),
an appropriate peptide spacer (linker) placement adjacent to IAV-derived peptides, and
a degron.

26. The polyepitope polypeptide of claim 25 further comprising a further five IAV-derived peptides that induce IFNγ by CD8+ T cells, wherein the further five IAV-derived peptides are SEQ ID NO:5 (GILGFVFTL), SEQ ID NO:3 (SRYWAIRTR), SEQ ID NO:16 (RRSGAAGAAVK), SEQ ID NO:2 (ELRSRYWAI), and SEQ ID NO:4 (CTELKLSDY).

27. The polyepitope polypeptide of claim 25, comprising at least 15 IAV-derived peptides capable of inducing IFNγ by CD8+ T cells selected from the group consisting of SEQ ID NO:1 (ILRGSVAHK), SEQ ID NO:2 (ELRSRYWAI), SEQ ID NO:3 (SRYWAIRTR), SEQ ID NO:4 (CTELKLSDY), SEQ ID NO:5 (GILGFVFTL), SEQ ID NO:6 (SIIPSGPLK), SEQ ID NO:7 (ASCMGLIY), SEQ ID NO:8 (FMYSDFHFI), SEQ ID NO:9 (FVRQCFNPM), SEQ ID NO:10 (VSDGGPNLY), SEQ ID NO:11 (FLKDVMESM), SEQ ID NO:12 (NMLSTVLGV), SEQ ID NO:13 (MMMGMFNML), SEQ ID NO:14 (YSHGTGTGY), SEQ ID NO:15 (HSNLNDATY), SEQ ID NO:16 (RRSGAAGAAVK), SEQ ID NO:17 (LLTEVETYV), SEQ ID NO:18 (MVLASTTAK), SEQ ID NO:19 (RGINDRNFW), and SEQ ID NO:20 (FLLMDALKL).

28. The polyepitope polypeptide of claim 25, comprising 20 IAV-derived peptides that induce IFNγ by CD8+ T cells, with amino acid sequences SEQ ID NO:1 (ILRGSVAHK), SEQ ID NO:2 (ELRSRYWAI), SEQ ID NO:3 (SRYWAIRTR), SEQ ID NO:4 (CTELKLSDY), SEQ ID NO:5 (GILGFVFTL), SEQ ID NO:6 (SIIPSGPLK), SEQ ID NO:7 (ASCMGLIY), SEQ ID NO:8 (FMYSDFHFI), SEQ ID NO:9 (FVRQCFNPM), SEQ ID NO:10 (VSDGGPNLY), SEQ ID NO:11 (FLKDVMESM), SEQ ID NO:12 (NMLSTVLGV), SEQ ID NO:13 (MMMGMFNML), SEQ ID NO:14 (YSHGTGTGY), SEQ ID NO:15 (HSNLNDATY), SEQ ID NO:16 (RRSGAAGAAVK), SEQ ID NO:17 (LLTEVETYV), SEQ ID NO:18 (MVLASTTAK), SEQ ID NO:19 (RGINDRNFW), and SEQ ID NO:20 (FLLMDALKL).

29. An antigenic or immunogenic formulation comprising the polynucleotide of claim 17.

30. A vaccine formulation produced by mixing the antigenic or immunogenic formulation of claim 29 with a pharmaceutically acceptable excipient.

31. A method of treating a subject to generate a humoral vaccine response toward an influenza virus hemagglutinin protein, the method comprising:
administering to the subject the vaccine formulation of claim 30 together with, in addition to, or concomitant with a vaccine directed at generating a humoral vaccine response toward an influenza virus hemagglutinin protein.

32. An antigenic formulation comprising the polyepitope polypeptide of claim 25.

33. An immunogenic formulation comprising the polyepitope polypeptide claim 25.

34. A vaccine formulation produced by mixing the immunogenic formulation of claim 33 with a pharmaceutically acceptable excipient.

35. A method of treating a subject to generate a humoral vaccine response toward an influenza virus hemagglutinin protein, the method comprising:
administering to the subject the vaccine formulation of claim 30 together with, in addition to, or concomitant with a vaccine directed at generating a humoral vaccine response toward an influenza virus hemagglutinin protein.

36. A polypeptide comprising:
influenza A virus (IAV)-derived peptides that induce IFNγ by CD8+ T cells, with amino acid sequences SEQ ID NO:1 (ILRGSVAHK), SEQ ID NO:2 (ELRSRYWAI), SEQ ID NO:3 (SRYWAIRTR), SEQ ID NO:4 (CTELKLSDY), SEQ ID NO:5 (GILGFVFTL), SEQ ID NO:6 (SIIPSGPLK), SEQ ID NO:7 (ASCMGLIY), SEQ ID NO:8 (FMYSDFHFI), SEQ ID NO:9 (FVRQCFNPM), SEQ ID NO:10 (VSDGGPNLY), SEQ ID NO:11 (FLKDVMESM), SEQ ID NO:12 (NMLSTVLGV), SEQ ID NO:13 (MMMGMFNML), SEQ ID NO:14 (YSHGTGTGY), SEQ ID NO:15 (HSNLNDATY), SEQ ID NO:16 (RRSGAAGAAVK), SEQ ID NO:17 (LLTEVETYV), SEQ ID NO:18 (MVLASTTAK), SEQ ID NO:19 (RGINDRNFW), and SEQ ID NO:20 (FLLMDALKL),
at least one linker comprising a tripeptide AAY, and
a degron comprising ubiquitin.
